(19) United States
(12) Patent Application Publication
(10) Pub. No.: US 2023/0101856 A1
(43) Pub. Date: Mar. 30, 2023

(54) NANOMATERIALS COATED WITH CALIXARENES

(71) Applicant: UNIVERSITE LIBRE DE BRUXELLES, Bruxelles (BE)

(72) Inventors: Gilles BRUYLANTS, Bruxelles (BE); Ivan JABIN, Bruxelles (BE); Maurice RETOUT, Bruxelles (BE); Bryan GOSSELIN, Bruxelles (BE)

(73) Assignee: UNIVERSITE LIBRE DE BRUXELLES, Bruxelles (BE)

(21) Appl. No.: 17/799,248

(22) PCT Filed: Feb. 10, 2021
(86) PCT No.: PCT/EP2021/053170

§ 371 Date: Aug. 11, 2022

(30) Foreign Application Priority Data
Feb. 11, 2020 (EP) 20156678.3
Nov. 3, 2020 (EP) 20205365.8

(51) Int. Cl. C07C 213/02

(52) U.S. Cl. CPC C07C 213/02; B82Y 5/00

(57) ABSTRACT

This invention concerns a versatile and simple one-pot method to prepare nanomaterials, and in particular nanoparticles, grafted with an ultra-thin layer of calixarenes by placing at 5 least one oxidized metal with at least one calix[n]arene diazonium salt in the presence of a reducing agent in a solvent, and heating the traction mixture to obtain a metal-based nanomaterial coated with calix[n]arenes. The invention further concerns the coupling of organic molecules or biomolecules to the calixarene-grafted nanomaterials in order to further functionalize the surface of the particles. The metal-based nanomaterial coated with 10 calix[n]arenes can for example be used in immunoassays.

### FIELD OF THE INVENTION

This invention concerns a versatile and simple one-pot method to prepare nanomaterials, and in particular nanoparticles, grafted with an ultra-thin layer of calixarenes as well as a new process for manufacturing these materials. The invention further concerns the use of these calixarene-grafted nanomaterials.

### BACKGROUND OF THE INVENTION

The use of nanomaterials, and in particular metal-based nanomaterials has become widespread in many technical fields, from biomedical applications or paints to microelectronics or as chemical catalysts. Their preparation is however not always straightforward, due to stability issues or difficulty to control their size and/or shape. Their functionalization is not simpler for the same stability reasons, and because reactive groups have to be introduced at the surface of the nanomaterial.

For example, silver nanoparticles are known to have anti-bacterial effect, which in part results from their instability and release of silver ions. This same instability prevents them from being functionalized. It is therefore so far extremely difficult to prepare functionalized silver nanoparticles.

One class of organic substances that has been proposed for immobilizing or grafting onto material surfaces is that of the calix[n]arenes (or calixarenes). Calix[n]arenes are cyclic phenoxy derivatives in which n is the number of phenoxy groups, linked in their ortho positions by methylene bridges or sulfur bridges in the case of thiacalixarenes. Calixarenes are conformationally flexible molecules that can display different conformations due to the ability to undergo complete ring inversions. Calixarenes can eventually possess a cup-like structure (i.e. cone conformation) having a narrow and a large rim.

In the following, the terms “calix[n]arenes” and “calixarenes” refer to both families of compounds, those with methylene bridges (named calix[n]arenes), and those with S bridges (named thiacalix[n]arenes) and their oxide derivatives (SO and SO₂ bridges). In addition, calix[n]arenes also have varying numbers of phenoxy moieties expressed by the symbol [n], wherein n represents the number of phenoxy moieties, in particular n can be 4, 5, or 6. Calix[n]arenes are known compounds that have been synthesized with various substitution patterns, for example with substituents on the aromatic part of the phenoxy moieties or on their hydroxyl groups. These cyclic compounds can find various applications in a manifold of areas, including the development of enzyme mimetics, ion sensitive electrodes or sensors, selective membranes, non-linear optics, and HPLC stationary phases.

Calix[n]arenes have been used as coatings on various materials. The immobilization of (thia)calixarenes onto a surface has been reported using different attachment techniques. The resulting immobilized calixarenes were used as receptors.

EP2836539 discloses a grafting method to coat an ultrathin layer of calixarenes on the surface of a material. The calixarenes are grafted via the large rim, using diazonium chemistry, and it leads to rigid and stable molecular layers, which offer a robust and stable platform for further (post-)functionalization. The macrocyclic structure of the calixarene prevents polymerization during the grafting process, induces spatial pre-organization and pre-structuration and allows the orthogonal polyfunctionalization of the platforms, with a precise spatial control.

Though leading to robust grafting, this method can only be efficient for stable nanomaterials, and could, for example not be applied to silver nanostructures.

There is a need for a more versatile and tunable method to prepare nanomaterials functionalized with calixarenes, with a highly robust, structurally regular, ultra-thin layer, in a controlled manner. Indeed, for certain applications, it is important to control the size and shape of the nanomaterial as well as the surface density, e.g. for applications as sensors, because this may have an impact on the detection sensitivity and efficiency.

### SUMMARY OF THE INVENTION

In accordance with the present invention, a one-pot synthesis gives nanomaterials grafted or coated with an ultra-thin layer of calix[n]arenes from a metallic salt (or a mixture of metallic salts) and a calixarene-diazonium salt (or a mixture of calixarene-diazonium salts), in reductive conditions. This leads to the formation of a robust nanomaterial coated with an ultra-thin layer, which may be rather dense and which, if desired, can serve as a platform for one or more further functionalization(s).

To this purpose, the invention relates to a method to synthesize metal-based nanomaterials coated with calix[n]arenes comprising the steps of:
placing at least one oxidized metal MX with at least one calix[n]arene-diazonium salt C—N₂ in presence of a reducing agent in a solvent, and
heating the reaction mixture to obtain a metal-based nanomaterial coated with calix[n]arenes MC.
   By at least one, it is referred to at least one type of.
   An oxidized metal refers to a metal with metal or metal derivative having a non-null oxidation state. Preferably, an oxidized metal refers to a metal having a positive oxidation state. It can be a metal salt or a metal oxide.
   The method offers access to robust and stable nanomaterials, which, depending on the type of calixarene used, can be further functionalized. The macrocyclic structure of the calixarene prevents polymerization during the synthesis process, induces spatial pre-organization and pre-structuration and allows the orthogonal polyfunctionalization of the platforms, with a control on the composition of the final layer.
   The reaction scheme is as follows:

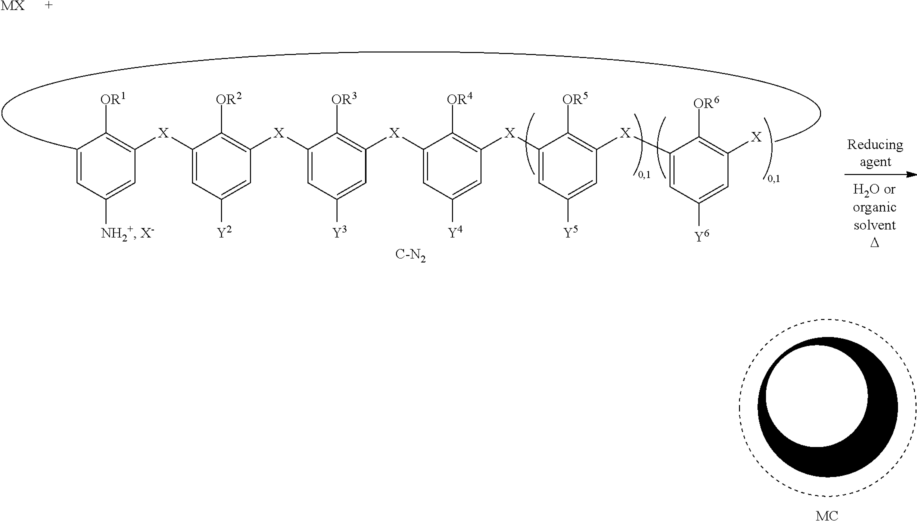

Using this method, the calix[n]arenes are bound onto the metallic surface of the nanomaterial with covalent bonds.
The method of the invention can further comprise the step of adjusting the value of the pH of the reaction mixture preferably between 3.5 and 10, preferably between 4.5 and 9.5, preferably between 5.5 and 9, preferably around 8,
The molar ratio of metal-based salt to calixarenes influences the number of particles formed and hence their size, as well as the quality of the coating. A too large excess of calixarene diazonium may lead to the calixarene reacting with itself. This ratio should preferably be fixed between 5:1 and 1:5, preferably between 4:1 and 1:4, preferably between 2:1 and 1:2, still preferably around 1:1.

The reducing agent can be any reducing agent found suitable by the person skilled in the art like for example hydrides (e.g. sodium borohydride, sodium cyanoborohydride, . . . ), ascorbate salts (e.g. sodium ascorbate), etc. A combination of reducing agents can also be used, in particular, for example a combination comprising ascorbate. The strength and the amount of reducing agent can be modified in order obtain different sizes: weaker reducing agents or lower amounts of it will lead to bigger nanoparticles. The molar proportion of reducing agent to calixarene-diazonium salt is preferably comprised between 20:1 and 1:10, preferably between 15:1 and 1:5, preferably between 10:1 and 1:4, preferably between 5:1 and 1:2, still preferably around 4:1. The person skilled in the art will know how to adapt proportions, taking for example into account the number of hydrides a given reducing agent can provide.

Heating is preferably performed under mixing, at temperatures comprised between 15° C. and 150° C., preferably between 20° C. and 120° C., preferably between 25° C. and 100° C., preferably between 40 and 80° C. and still preferably around 60° C. It was observed by the applicants that the reaction temperature has an impact on the particle size distribution.

A reaction temperature of around 60° C. leads to a narrow size distribution.

Reaction time is at least 1 h, preferably at least 2 h or at least 3 h, still preferably at least 5 h or at least 8 h and up to 48 h, up to 24 h, preferably up to 16 h. The applicant has noted that after 16 h, the formation of the nanoparticles was completed as well as the grafting of the calixarenes onto these particles.

The invention also relates to the metal-based nanomaterials coated with calix[n]arenes obtained by the process of the invention, which have the distinctive features over nanomaterials obtained by other methods to have an homogeneous particles distribution (i.e. above 80% of the particle formed have the same average dimension ±10%). Moreover, the metal-based nanomaterials of the invention are coated only with calix[n]arenes, meaning that the only ligands at the surface of the metal are calixarenes. Indeed, with other methods, where a ligand like citrate, present at the surface of a particle, is interchanged with a calixarene, there are always some residual citrate ligands at the surface of the metal. The metal-based nanomaterials of the invention can therefore be distinguished from metal-based nanomaterials prepared by ligand-exchange techniques by the absence of residual ligands other than calixarenes at their surface.

Preferably, the oxidized metal MX is a metal oxide or a mixture of metal oxides, or a salt of a metal or a mixture of salts.

The salt generally implies that the metallic part is positively charged, the counter ion can be any suitable ion. The metal-based salt can for example be a halide, a nitrate, a sulfate, a carboxylate, a triflate, an alcoolate, an hydroxylate, a sulfonate, a phosphate (like hexafluorophosphate), a tosylate, a borate (like tetrafluoroborate) . . . . In some cases, the salt can be not clearly ionized, such as for example silicate organic salts like tetraethyl orthosilicate (TEOS), trimethyl orthosilicate (TMOS), . . . .

An halide is preferably a chloride, a fluoride, a bromide or an iodide.

The oxidized metal is not necessarily soluble in water. For example, hexafluorophosphate salts or tetrafluoroborate salts can be suitable in organic solvents.

A carboxylate can be any suitable carboxylate ion, derived from a carboxylic acid, preferably a C₁-C₂₀, still preferably a C₁-C₁₀, branched or unbranched, substituted or unsubstituted, carboxylic acid and more preferably, the carboxylate is formate, acetate, propionate, butyrate, lactate, oxalate, citrate, trifluoroacetate or oxalate.

A metal can be any of the metals as defined in the periodic table of the elements, an in particular of the subclasses alkali metals, alkaline earth metals, lanthanides, actinides, transition metals, post transition metals, or metalloids. Preferably, the metal is a transition metal or a post transition metal. Preferably, the metal is selected from the list comprising silver, palladium, gold, platinum, copper, nickel, zinc, cadmium, indium, lead, aluminum, titanium, silicon, tantalum or iron.

A metal oxide is any oxide of the metals as defined above. Preferably, a metal oxide is a transition metal oxide, such as, but not limited to, tisane oxide, tantalum oxide, iron oxide, copper oxide, silver oxide, nickel oxide or a post transition metal oxide, such as, but not limited to, silicon oxide, zinc oxide, cadmium oxide, indium oxide, lead oxide or aluminum oxide.

An alloy or mixture of metals is a combination of two or more metals and/or metal oxides as defined above. In some cases, an alloy comprises at least two metals and/or metal oxides from the same subclasses or from different subclasses. Preferably, an alloy comprises at least two metals or metal oxides from the subclass transition metal and/or post transition metal. Preferably, the alloy comprises at least two metals and/or metal oxides selected from the list comprising silver, palladium, gold, platinum, copper, nickel, zinc, cadmium, indium, lead, aluminum, iron, titanium, silicon, tantalum, and their respective oxides. A metal base salt is for example silver nitrate, palladium dichloride, platinum dichloride, chloroauric acid, copper (II) acetate, iron (II) chloride, iron (III) chloride.

A nanomaterial according to the present invention is a particulate material (nanoparticle) having at least one dimension in the nanometric range, i.e. between 1 and 999 nm, preferably between 5 and 800 nm, still preferably between 10 and 500 nm, preferably below 250 nm or below 150 nm or below 100 nm as generally accepted for a nanomaterial. A nanomaterial according to the present invention can have any shape, like for example, but not limited to a spherical shape, a cubical shape, a star shape, a rod shape, a wire shape, a nanocage or a triangular shape. The final shape of the nanomaterial is dependent on the nature of the metal-based element(s), the reacting conditions like pH, nature of the reducing agent, temperature, addition rate of the reducing agent, . . . .

The metal-based nanometarial core can be designed to have various properties by including one or more elements conferring for example optical properties (with e.g. gold, silver, copper, platinum), magnetic properties (with e.g. iron oxide), catalytic properties (with e.g. copper, palladium, titanium, platinum, tantalum) or antimicrobial properties (with e.g. silver or copper).

Calix[n]arenes are organic macrocycles wherein four or more phenolic structures are linked so as to form a crown, the linker between the phenolic structures usually consisting of CH₂, S, SO or SO₂. n designs the number of phenolic structures comprised in the macrocycle. n is typically comprised between 4 and 6.

When present on a nanomaterial surface, each of the aromatic subunits of the calixarene can adopt either an “up” or a “down” orientation towards the grafted surface. “Up” orientation refers to the phenolic groups pointing in the direction of the surface and “down” orientation refers to the phenolic groups pointing in the opposite direction, away from the surface.

A calix[n]arene diazonium salt designs a calix[n]arene wherein at least one phenolic ring is functionalized with a diazonium salt, as represented in formula I.

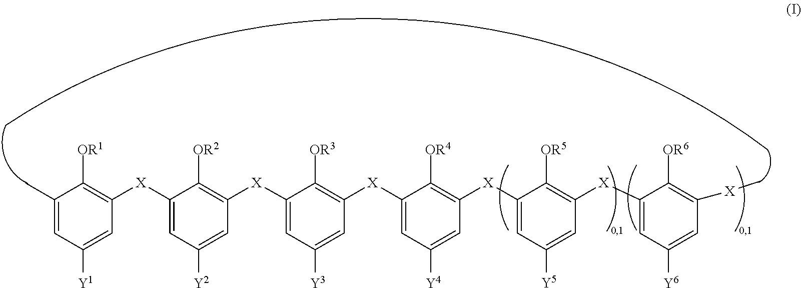

In one embodiment:

X represents CH₂, S, SO or SO₂; when X is S, the calixarene can be called a thiacalixarene;

R¹, R², R³, R⁴, R⁵ and R⁶ each independently represent hydrogen or C₁₋₃₀ alkyl optionally substituted with one or more substituents each independently selected from the group consisting of halo (e.g. fluoro, chloro, bromo, iodo), carboxylic acid ester, alkyl or benzyl thioester, alkenyl, alkynyl, C₁₋₃₀ alkoxy, aryl, substituted aryl (wherein the substituent is fluoro or cyano or C₁₋₃₀ alkyl or C₁₋₃₀ alkoxy), —N₃, cyano, carboxylic acid, carboxylic acid amide, —OH, amino, amido, imino, carbamate, acyl chloride, ureido, thioureido, mercapto, substituted disulfide, maleimide, heterocyclic, amino acid and amino acid derivative, peptide, protein, DNA, RNA, microRNA, phosphine or phosphine oxide, crown ether, aza-crown ether, cryptand, porphyrin, calixarene, cyclodextrin, resorcinarene, saccharide, and polyethylene glycol; and wherein two or more of R¹, R², R³, R⁴, R⁵ and R⁶ may be covalently linked either directly or by a bridge that includes oxygen, phosphine, phosphine oxide, sulfur, SO, SO₂, amino, imino, amido, ureido, thioureido, ester, thioester, alkene, alkyne or alkyl;

Y²—, Y³—, Y⁴— Y⁵— and Y⁶— are each independently selected from the group consisting of hydrogen, diazonium salt, OH, NO₂, halogen, C₁₋₃₀ alkyl, acyl, carboxylic acid and derivatives (e.g. ester, amide), —N₃ alkenyl or alkynyl;

Y¹— is a diazonium salt N₂⁺X⁻ where X⁻ represents an anion such as but not limited to BF₄⁻, PF₆⁻, Cl⁻, TsO⁻ (tosylate).

The index “0,1” at the right side of the aryl moieties bearing R⁵ and R⁶ in formula I means “0 or 1”, meaning that these aryl moieties, each independently, can be present or absent.

The synthesis of calix[n]arene-diazonium salts can be performed either by in situ diazotation of the amino groups or by diazotation followed by isolation of the diazonium salts of formula I, according to procedures known in the art, as for example disclosed in EP2836539.

The calix[n]arenes bound to the surface of the metal-based nanomaterial is at least one of the compounds of formula (II) wherein:

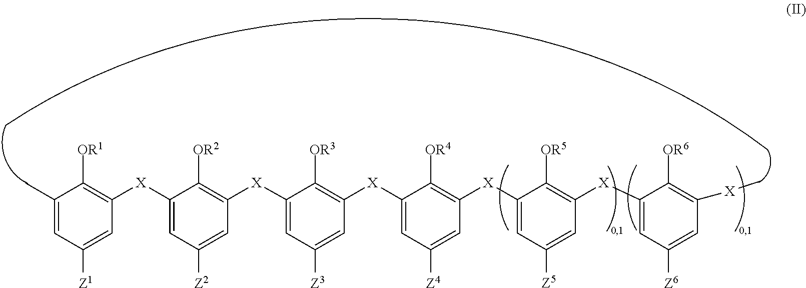

X represents CH₂, S, SO or SO₂;

R¹, R², R³, R⁴, R⁵ and R⁶ each independently represent hydrogen or C₁₋₃₀ alkyl optionally substituted with one or more substituents each independently selected from the group consisting of halo (e.g. fluoro, chloro, bromo, iodo), carboxylic acid ester, alkyl or benzyl thioester, alkenyl, alkynyl, C₁₋₃₀ alkoxy, aryl, substituted aryl (wherein the substituent is fluoro or cyano or C₁₋₃₀ alkyl or C C₁₋₃₀ alkoxy), —N₃, cyano, carboxylic acid, carboxylic acid amide, —OH, amino, amido, imino, carbamate, acyl chloride, ureido, thioureido, mercapto, substituted disulfide, maleimide, heterocyclic, amino acid and amino acid derivative, peptide, protein, DNA, RNA, microRNA, phosphine or phosphine oxide, crown ether, aza-crown ether, cryptand, porphyrin, calixarene, cyclodextrin, resorcinarene, saccharide, and polyethylene glycol; and wherein two or more of R¹, R², R³, R⁴, R⁵ and R⁶ may be covalently linked either directly or by a bridge that includes oxygen, phosphine, phosphine oxide, sulfur, SO, SO₂, amino, imino, amido, ureido, thioureido, ester, thioester,alkene, alkyne or alkyl;

Z²—, Z³—, Z⁴—, Z⁵— and Z⁶— are each independently selected from the group consisting of a covalent bond as defined for Z¹, hydrogen, diazonium salt, OH, NO₂, halogen, C₁₋₃₀ alkyl, acyl, carboxylic acid and derivatives (e.g. ester, amide), —N₃ alkenyl or alkynyl;

Z¹— is a covalent bond with the metal of the nanomaterial surface, said bond being a direct or indirect covalent bond.

A direct covalent bond, between the aromatic ring and the metal of the nanomaterial core may be obtained by loss of N₂ during the synthesis process. An indirect covalent bond may be obtained when N₂ is not lost during the synthesis process and remains as a linker between the aromatic ring and the metal.

The formation of a direct or indirect covalent bond may depend on the nature of the metal, the nature of the calixarene and/or the synthetic conditions.

It will be appreciated that groups having similar references in formula (I) and formula (II) are indeed similar, the nanomaterial of formula (II) resulting from the reaction of at least one of the diazonium salts of formula (I) according to the method of the invention. The following part of the present description will therefore refer to both compounds of formula (I) and compounds of formula (II). Chemical groups differing between the diazonium salt of formula (I) and the nanomaterial of formula (II) are noted differently.

Also preferably, at least one of Y²—, Y³—, Y⁴— Y⁵— and Y⁶— (in addition to Y¹—) is a diazonium salt.

Conversely, at least one of Z²—, Z³—, Z⁴— Z⁵— and Z⁶ is a bond with the metal of the nanomaterial surface.

Possibly, the calix[n]arene of formula I bears one, two, three, four or, if applicable, five or six diazonium groups, and an equal number of bonds are formed with the surface of the material. This may be applicable for the less flexible calix[n]arenes, in particular where n is 4, or with appropriately substituted calix[n]arenes. This may also be applicable for the more flexible calix[n]arenes, such as the calix[5]arenes or the calix[6]arenes which can be modified by adding appropriate substituents on the small rim or covalent bridges between the phenolic moieties (i.e. where two or more of R¹, R², R³, R⁴, R⁵ and R⁶ are covalently linked either directly or through a bridge as defined above).

The skilled person will be able to select the number of bonds to the surface per calixarene moiety based on the conformational flexibility of the (thia)calix[n]arene moiety, or on the possibilities in terms of chemical configuration, in particular as regards steric hindrance.

Preferably, the calix[n]arene is a calix[4]arene.

Preferably, the calix[n]arene is a calix[4]arene wherein at least one of Y²—, Y³— and Y⁴— is a diazonium salt, preferably at least two of Y²—, Y³— and Y⁴— are diazonium salts, preferably Y²—, Y³— and Y⁴— are diazonium salts.

Preferably, R¹, R², R³, R⁴, R⁵ and R⁶ each independently represent a C₁₋₃₀ alkyl optionally substituted with one or more R substituents each independently selected from the group consisting of C₁₋₃₀ alkoxy, —N₃, cyano, carboxylic acid, carboxylic acid amide, acid halide, amide ester, —OH, amino, amido, imino, carbamate, maleimide, thiol, cyanate, isocyanate or acyl chloride.

Preferably, R¹, R², R³, R⁴, R⁵ and R⁶ each independently represent polysaccharides, CH₂COOH, CH₂(CH₂OCH₂)ₘCH₂OCH₃, CH₂(CH₂OCH₂)ₚCH₂OCH₂COOH or CH₂(CH₂OCH₂)_{q}CH₂R, wherein R is as defined above, wherein m, p and q are not limited but are, each independently, preferably 50 or below.

The metal-based nanomaterial MC of the invention is coated with calix[n]arenes, meaning that a particulate nanomaterial is formed with a metallic central core M having calix[n]arenes bound to its surface so as to form an ultrathin layer C, as illustrated in Formula III.

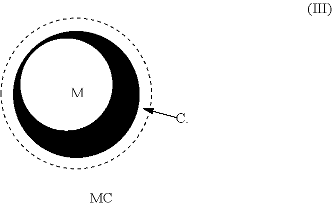

The ultrathin layer of calix[n]arenes is preferably a monolayer, meaning that the thickness of the ultrathin monolayer is the thickness of a calix[n]arene, including its substituents and functional groups.

Combinations of metal-based salts can be used, leading to the formation of mixed metal-based central cores, i.e. metal alloys.

Combinations of calix[n]arenes-diazonium salts can be used, leading to the formation of mixed layers whose composition can be controlled. As calix[n]arenes can be further functionalized, depending on the nature of R¹, R², R³, R⁴, R⁵ and R⁶, this presents the advantage of offering the possibility to confer a plurality of functionalities to the metal-based nanomaterials coated with calix[n]arenes.

The invention also relates the metal-based nanomaterials of the invention for use in immunoassays. The immunoassays are preferably immunoturbidimetry tests or lateral flow immune-assays (LFIAs).

The metal-based nanomaterials of the invention for use in immunoassays are preferably silver based and/or gold-based nanomaterials. Still preferably, the metal-based nanomaterials of the invention for use in immunoassays is a silver-based nanomaterial. So far, silver-based nanomaterials were usually not stable enough to be used in such kind of applications. Thanks to the method of the invention, this problem is overcome, and silver nanoparticles coated with calixarenes can be reliably produced and conjugated with biomolecules. The sensitivity of an immunoassay using silver based calixarene coated nanomaterials has been found to be much higher that the sensitivity of the same using gold.

The metal-based nanomaterials of the invention for use in immunoassays can be used to detect anti-SARS-CoV-2 human IgG (i.e. anti-SARS-CoV-2 human IgG: Anti-Spike-RBD fully human mAb(IgG )) and/or IgM and/or the viral Protein S (i.e. Recombinant SARS-CoV-2, S1 Subunit Protein (RBD)).

The metal-based nanomaterials coated with calix[n]arenes of the invention for use in immunoassays are preferably coated with calix[4]arenes.

The metal-based nanomaterials coated with calix[n]arenes of the invention for use in immunoassays are preferably bio-conjugated with a biomolecule, like a protein, the protein being bound to at least one of R¹, R², R³, R⁴, R⁵ and R⁶. The protein is preferably an immunoglobulin or any protein to be detected.

Bioconjugation can be done using various classical techniques, including, but not limited to, passive adsorption, peptide coupling, copper(I)-catalyzed alkyne-azide cycloaddition (CuAAC), or others (maleimide-thiol, hydrazine-aldehyde, . . . ) . . . .

### DETAILED DESCRIPTION OF THE INVENTION

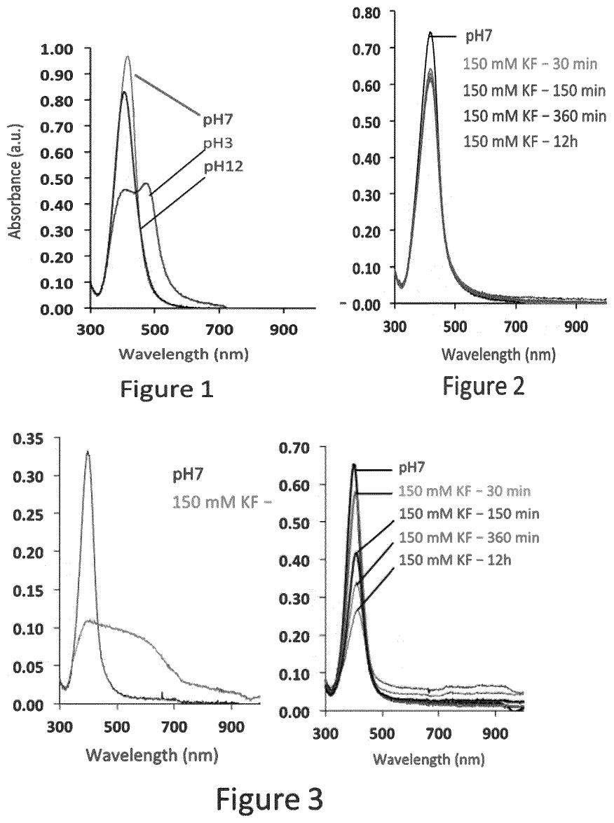

FIG. 1: UV-visible spectrum of Ag-C1-20 at various pH;

FIG. 2: UV-visible spectrum of Ag-C1-20 in presence of KF over time;

FIG. 3: UV-visible spectrum of Ag-citrate (left) and Ag—S-PEG (right) at pH 7 and in presence of KF;

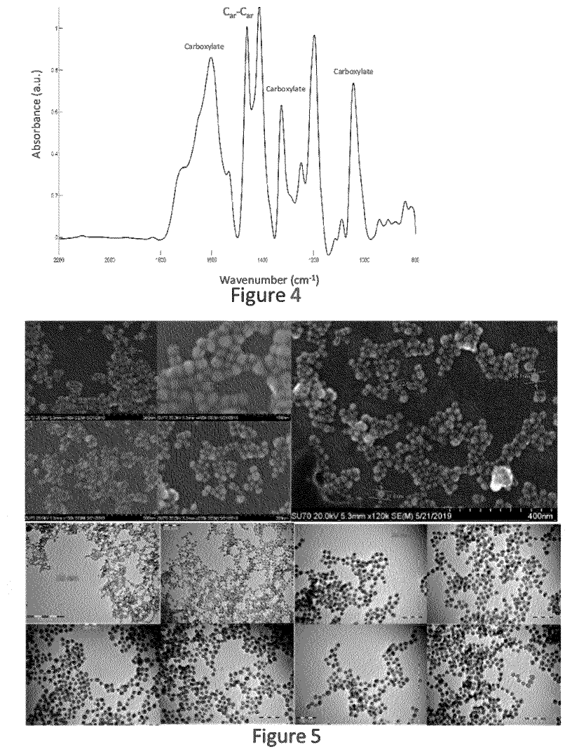

FIG. 4: IR spectrum of Ag-C1-20;

FIG. 5: TEM images of Ag-C1-20;

FIG. 6: UV-visible spectrum of particles obtained in example 2a, 2b, 2c;

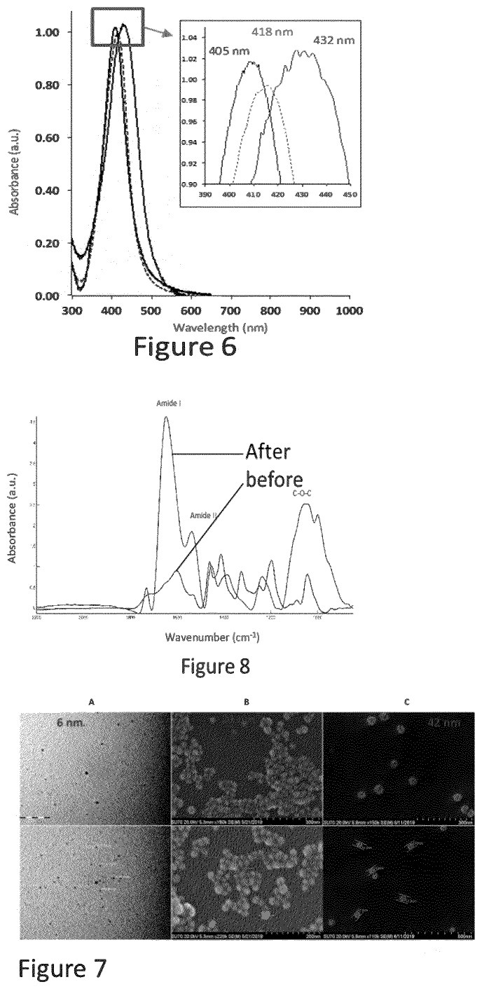

FIG. 7: TEM images of particles obtained in example 2a, 2b, 2c;

FIG. 8: IR spectrum of Ag—C1 post functionalized;

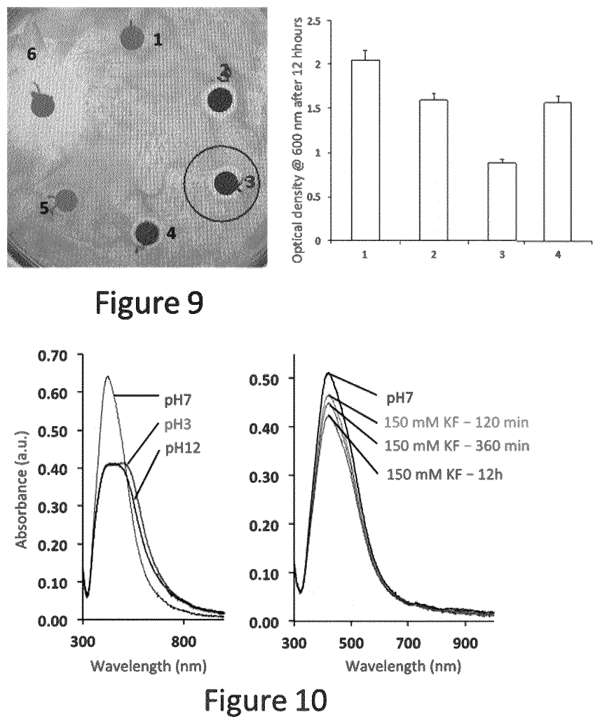

FIG. 9: E. Coli growth inhibition study by disc diffusion method for different conditions (1=water; 2=Ag-citrate; 3=Ag—C1; 4=Ag—C2; 5=60 μM Kanamycine; 6=60 mM Kanamycine) and right: optical density at 600 nm of a suspension of bacteria after 12 hours in different conditions (same numbering of the samples as for the disc diffusion method);

FIG. 10: UV-visible spectrum of Ag—C2 at various pH (left) and in presence of KF over time (right);

FIG. 11: IR spectrum of Ag—C2;

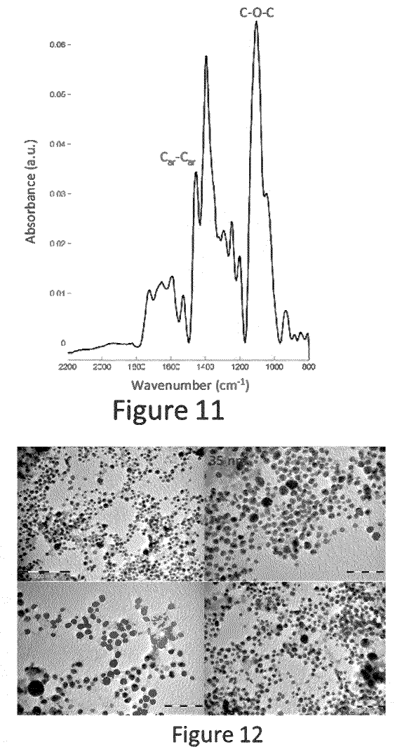

FIG. 12: TEM images of Ag—C2;

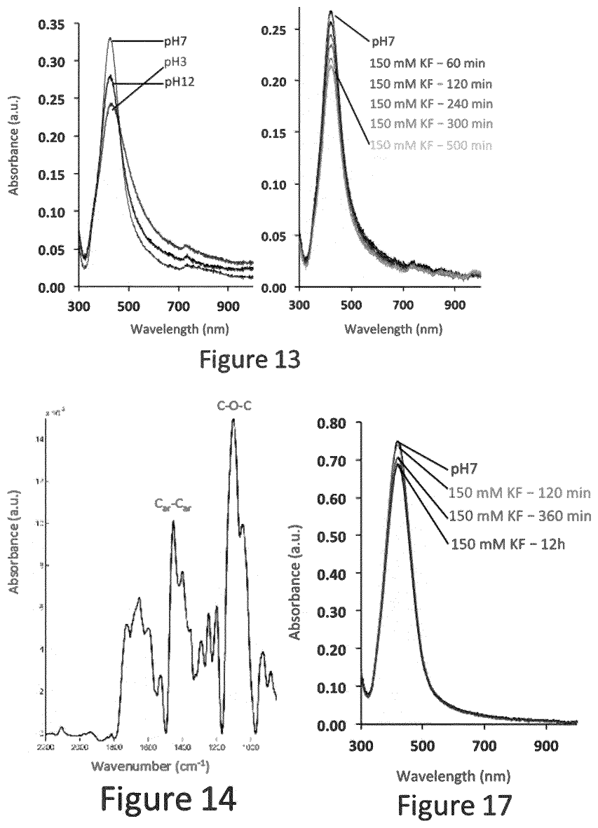

FIG. 13: UV-visible spectrum of Ag—C3 at various pH (left) and in presence of KF over time (right);

FIG. 14: IR spectrum of Ag—C3;

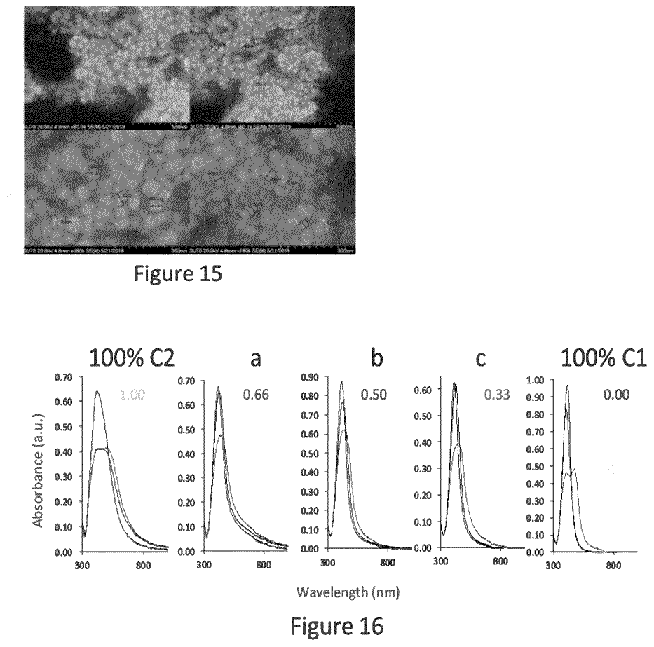

FIG. 15: TEM images of Ag—C3;

FIG. 16: UV-visible spectrum of Ag—C1C2, for various ratio of C1 to C2, at various pH;

FIG. 17: UV-visible spectrum of Ag—C1C2, for a ratio of C1 to C2 of 50:50, in presence of KF over time;

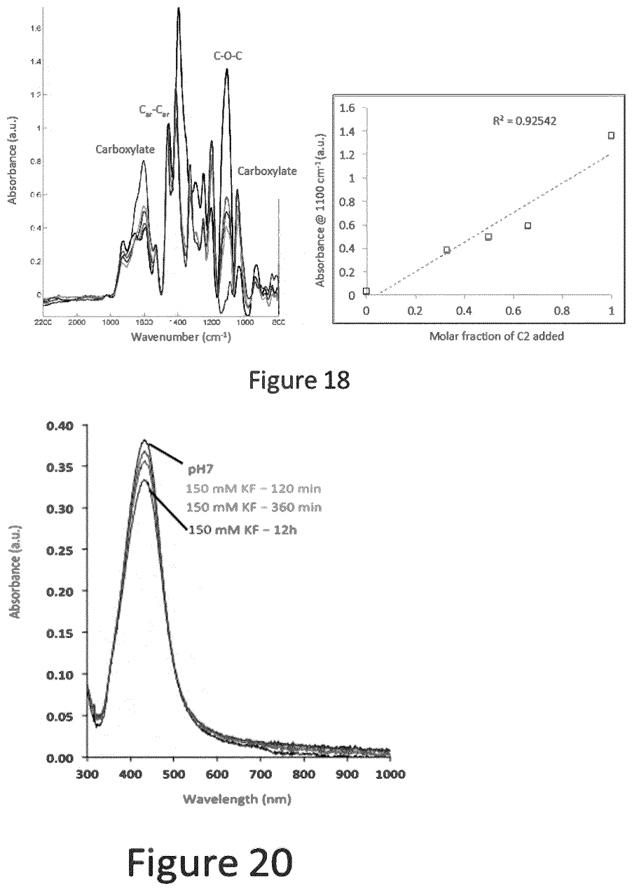

FIG. 18: IR spectrum of Ag-C1C2, for various ratio of C1 to C2, overlaid;

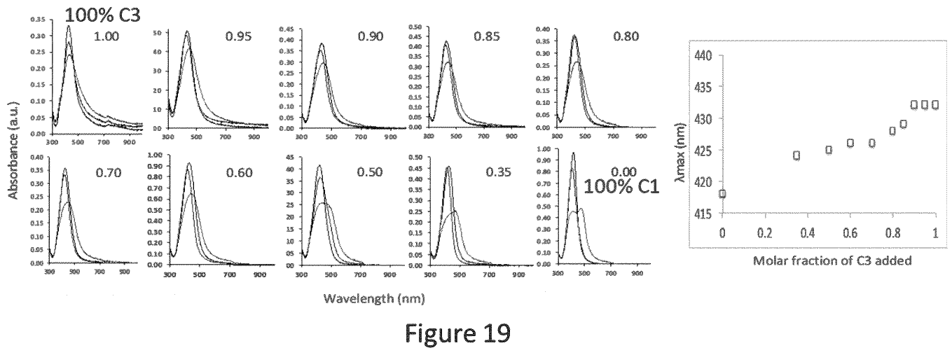

FIG. 19: UV-visible spectrum of Ag-C1C3, for various ratio of C1 to C3, at various pH, and graph displaying the shift of absorption peak in function of ratio of C3;

FIG. 20: UV-visible spectrum of Ag-C1C3, for a ratio of 85% C3, in presence of KF over time;

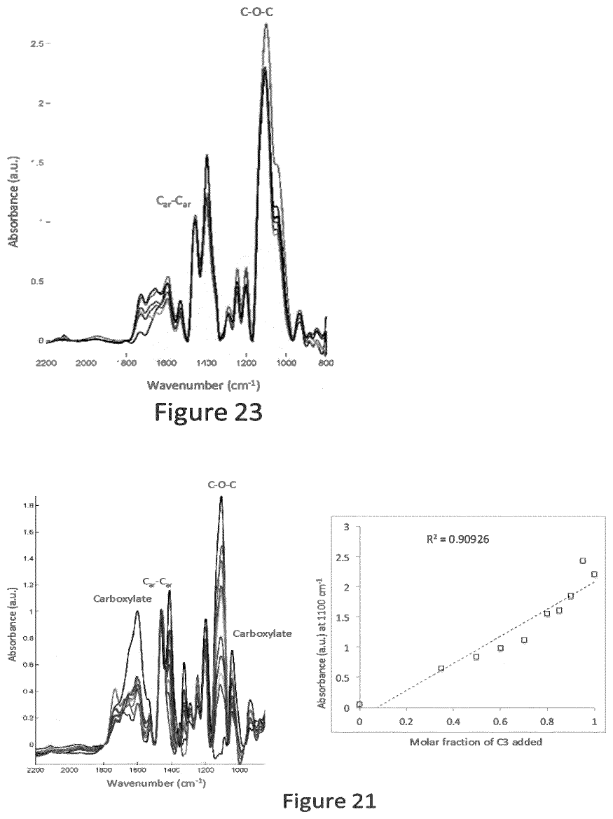

FIG. 21: IR spectrum of Ag-C1C3, for various ratio of C1 to C3, overlaid;

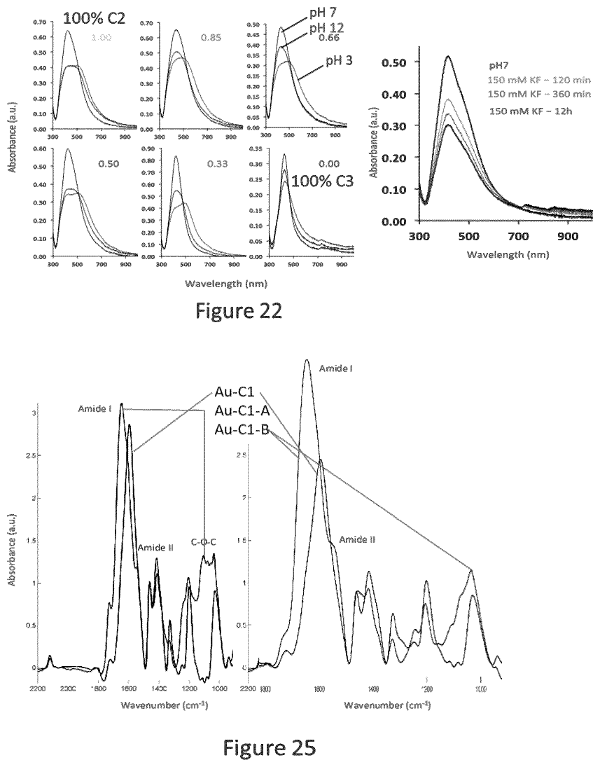

FIG. 22: UV-visible spectrum of Ag—C2C3, for various ratio of C2 to C3, at various pH, UV-visible spectrum of Ag—C2C3, for a ratio of 50% C3, in presence of KF over time;

FIG. 23: IR spectrum of Ag—C2C3, for various ratio of C2 to C3, overlaid;

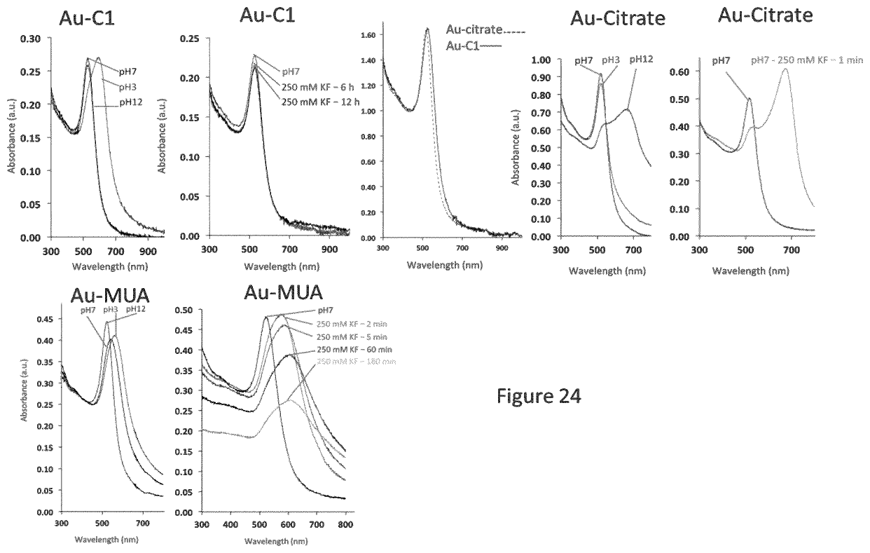

FIG. 24: UV spectrum of Au—C1 at various pH, and in presence of KF over time, compared with spectrum in the same conditions for Au-citrate and Au-MUA;

FIG. 25: IR spectrum of Au—C1, before and after functionalization with NH2-PEG7-OCH3 (Au-C1-A) and peptide of sequence AAPLSQETFSDLWKLL (Au-C1-B);

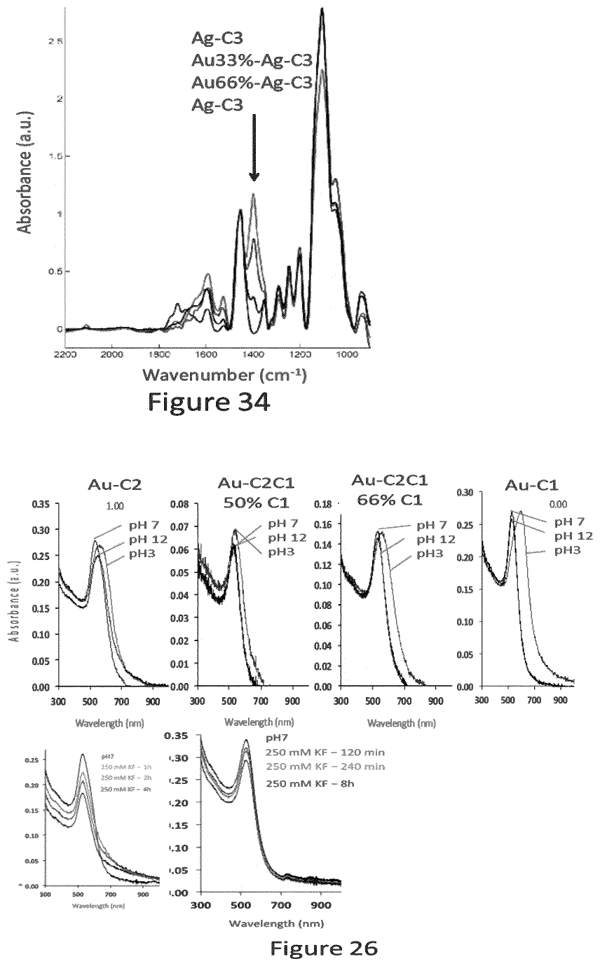

FIG. 26: UV-visible spectrum of Au-C2C1, for various ratio of C2 to C1, at various pH, UV-visible spectrum of Au-C2C1, for ratios of 0% and 50% C1, in presence of KF over time;

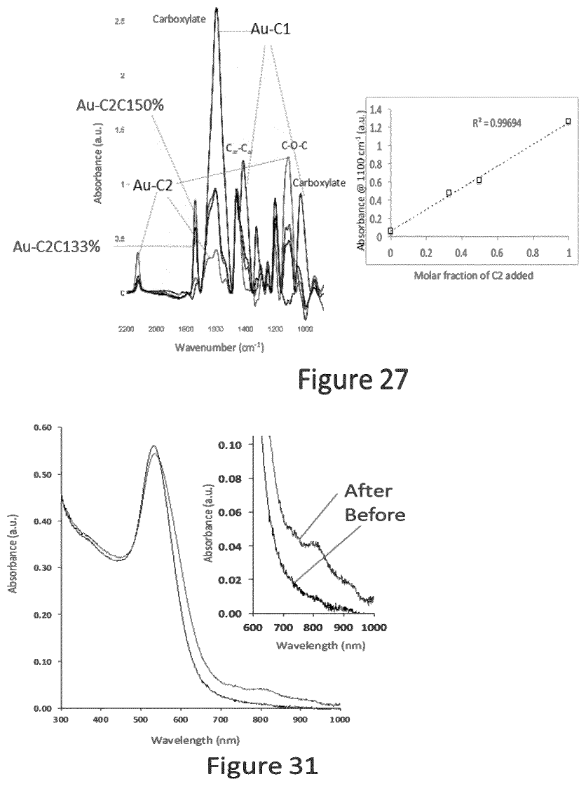

FIG. 27: IR spectrum of Au-C1C2, for various ratio of C1 to C2, overlaid;

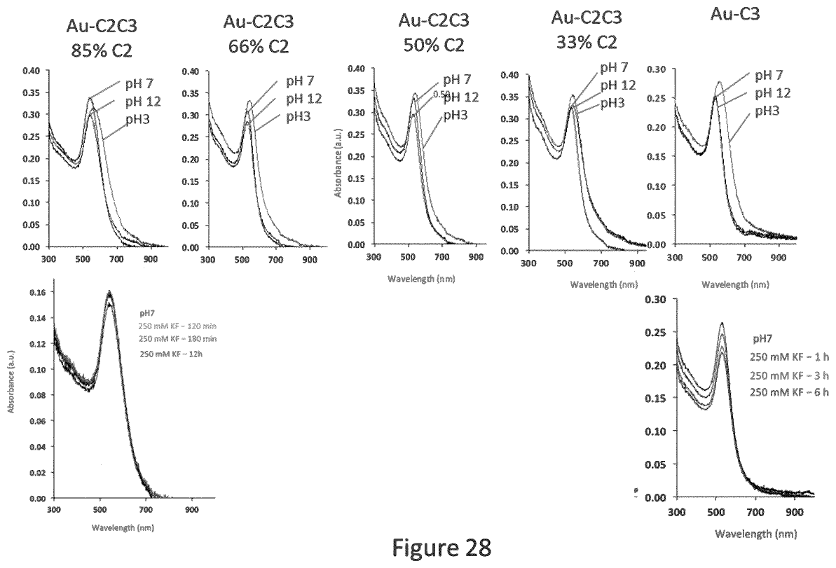

FIG. 28: UV-visible spectrum of Au—C2C3, for various ratio of C2 to C3, at various pH, UV-visible spectrum of Au—C2C3, for ratios of 0% and 85% C2, in presence of KF over time;

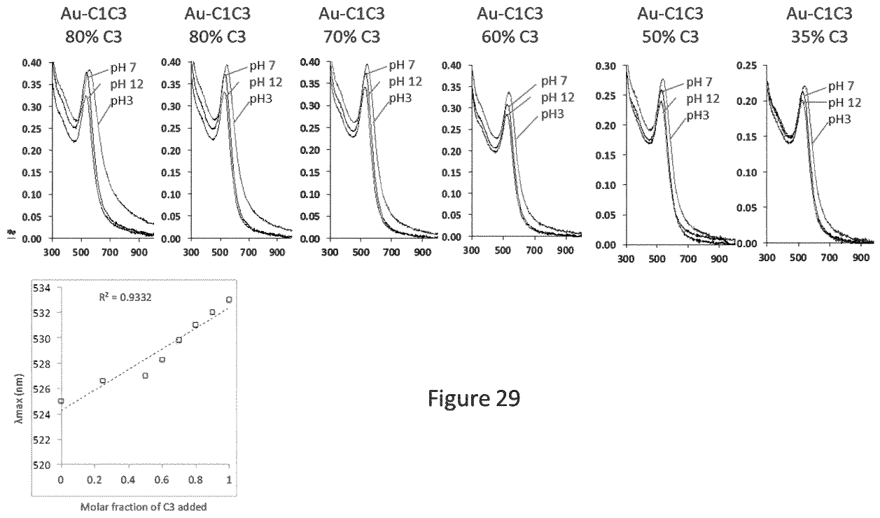

FIG. 29: UV-visible spectrum of Au—C1C3, for various ratio of C1 to C3, at various pH, and graph displaying the shift of absorption peak in function of ratio of C3;

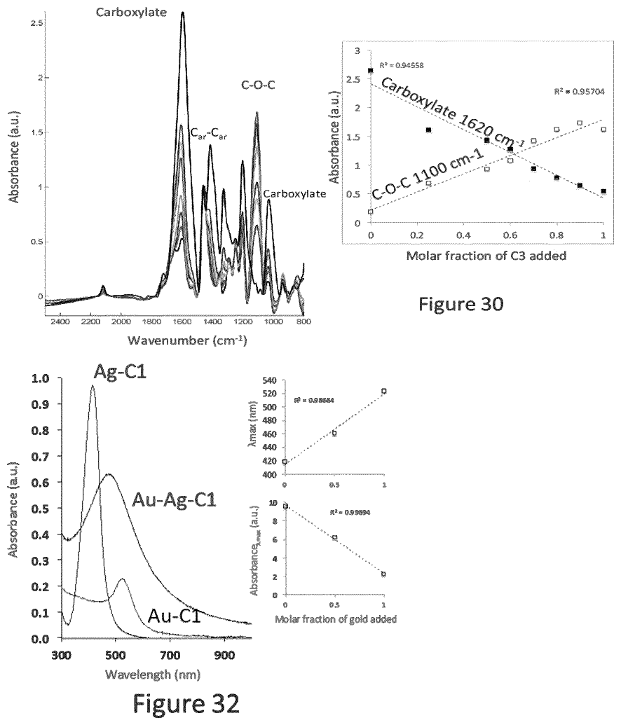

FIG. 30: IR spectrum of Au—C1C3, for various ratio of C1 to C3, overlaid, and graph displaying the evolution of selected peaks intensity in function of ratio of C3;

FIG. 31: IR spectrum of Au—C1C3 with 60% C3, before and after functionalization with Cyanine dye with terminal amino group absorbing at 800 nm;

FIG. 32: UV spectra of Ag—C1, Au—C1 and AuAg—C1;

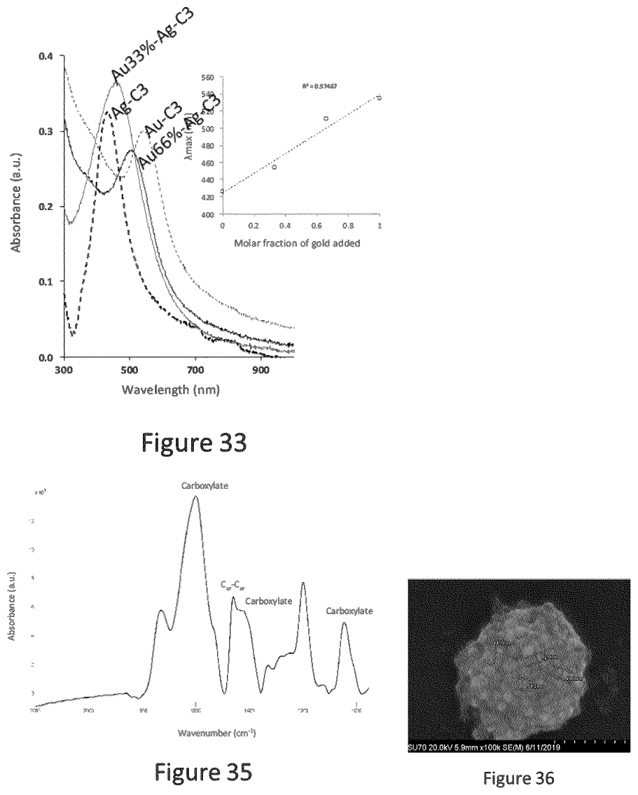

FIG. 33: UV spectra of Ag—C3, Au—C3 and AuAg—C3 (33% Au and 66% Au);

FIG. 34: overlaid IR spectrum of Ag—C3, Au—C3 and AuAg—C3 (33% Au and 66% Au);

FIG. 35: IR spectrum of Pd—C1;

FIG. 36: SEM image of Pd—C1;

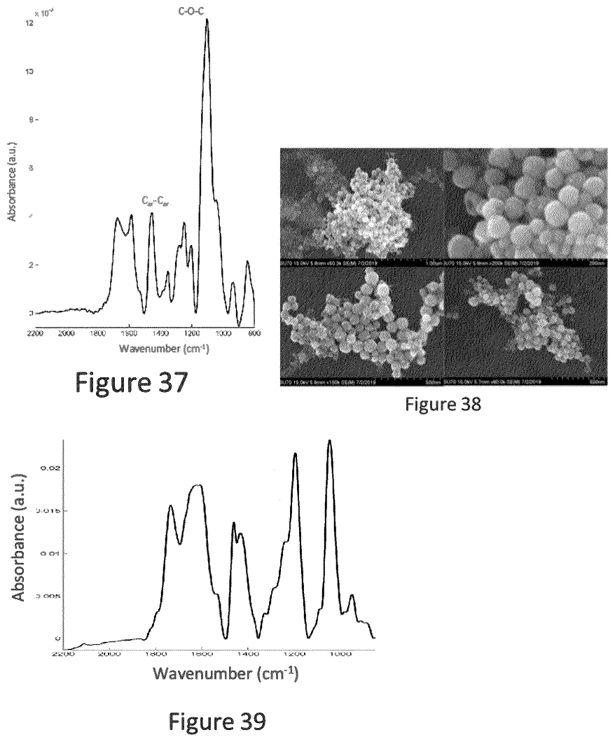

FIG. 37: FIG. 35: IR spectrum of Pd—C3;

FIG. 38: TEM image of Pd—C3;

FIG. 39: IR spectrum of Pt—C1;

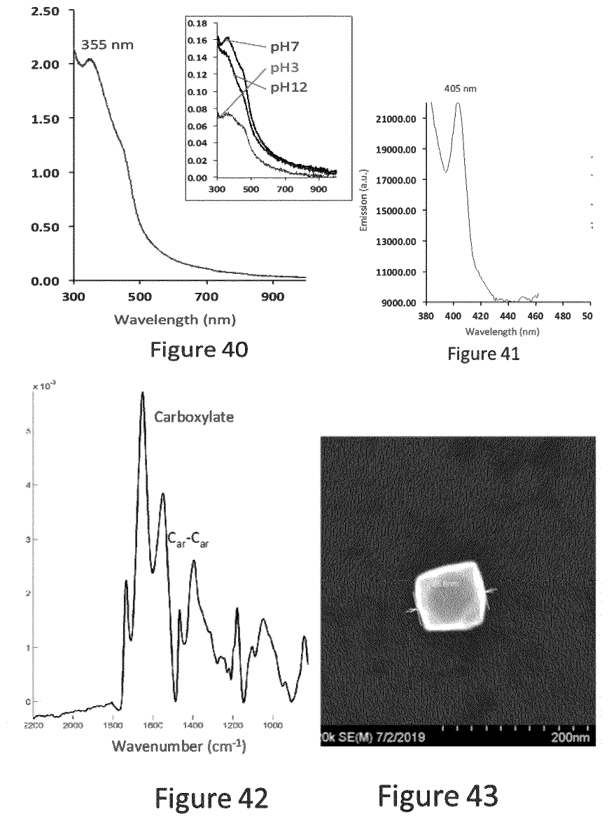

FIG. 40: UV spectrum of Cu—C1 at various pH;

FIG. 41: florescence emission spectrum of Cu—C1 demonstrating presence of copper oxide particles;

FIG. 42; IR spectrum of Cu—C1;

FIG. 43: SEM image of Cu—C1;

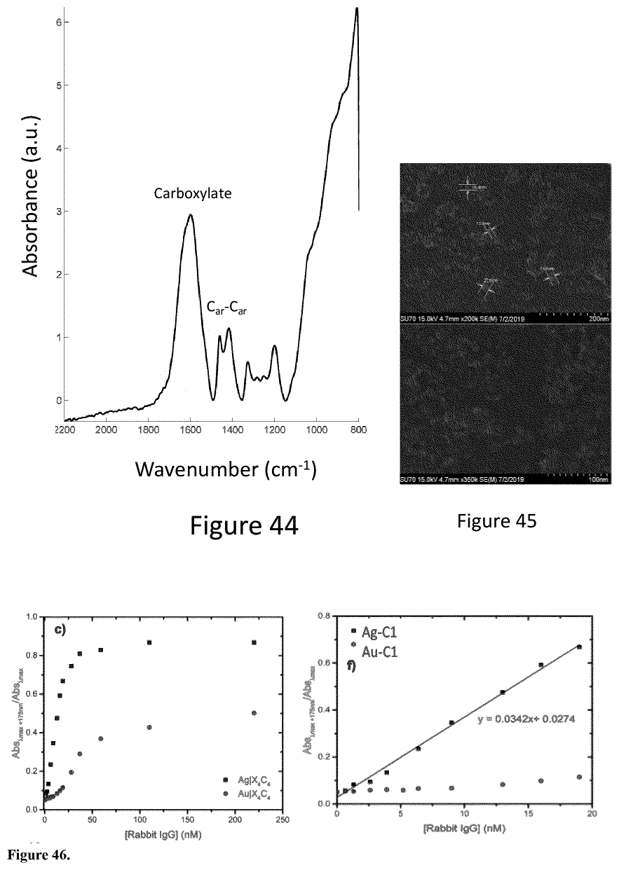

FIG. 44: IR spectrum of Fe—C1

FIG. 45: SEM images of Fe—C1;

FIG. 46. (c, f) Ratio between the absorbance of aggregated nanoparticles (λmax+175 nm) and the absorbance of dispersed nanoparticles (λmax) as a function of Rabbit IgG concentration for Au—C1 and Ag-C1-20;

FIG. 1. UV-Vis spectra of Ag—C1-20 and Ag-C1-20-Anti-human-IgG (left) as well as the corresponding IR spectra (right);

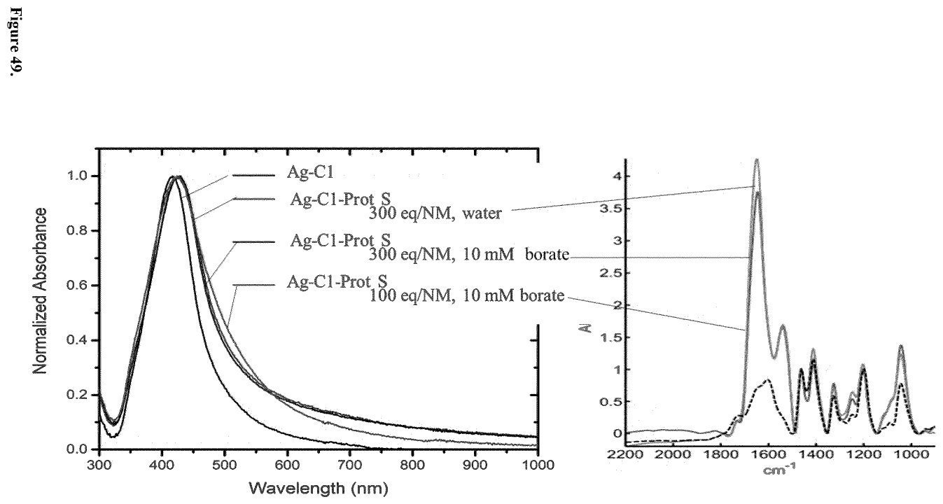

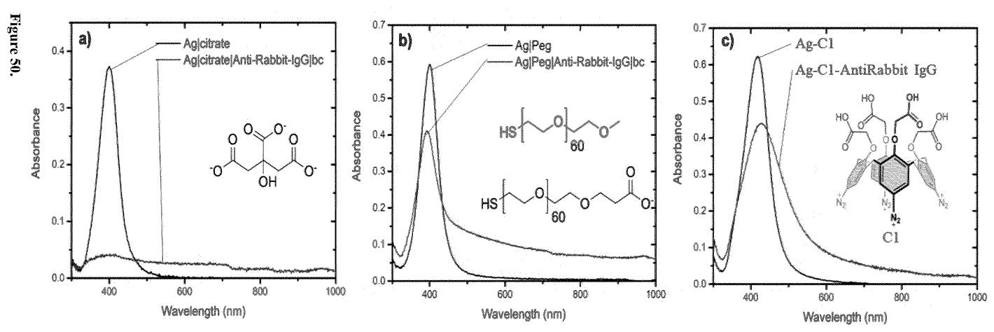

FIG. 48. UV-Vis spectra of Au-C1-Anti-human-IgG, Au-C1-Anti-human-IgM|ad and Au—C1-Prot-S|ad;

FIG. 49. UV-Vis spectra of Ag—C1 and Ag-C1Prot-S|ad synthesized in the indicated conditions (left). Corresponding IR spectra (right);

FIG. 50. UV-Vis spectra of Ag|itrate (a), Ag|Peg (b) and Ag—C1 (c) before and after bioconjugation of anti-Rabbit IgG with EDC/NHS;

FIG. 2. UV-Vis spectra of Ag—C3 C2 before and after bioconjugation with protein S or Anti-Human-IgG. (b) IR spectra of Ag—C3C2, Ag-C3C2-Prot-S1-bc pc and Ag-C3C2-anti-Human-IgG-bcpc.

FIG. 52. detection procedure

FIG. 3. (a) Scheme of the biomolecules involved in the detection test. Dashed arrows show the interactions between the biomolecules. (b) Scheme of the nanoparticles used for the detection test. (c) Scheme of the detection procedure: (1) Strip used for the vertical flow assay, (2) Incubation of the strip with human serum mixed with nanoparticles and liquid migration and (3) immobilization of the nanoparticles on the strip in the presence of a Anti-Sars-CoV-2 human IgG directed towards the viral S-protein.

FIG. 54. Images of the strips incubated with different concentrations of AgX4C4Ag-C1-Anti-Human-IgG_ad or Au-Citrate-Anti-Human-IgG_ad.

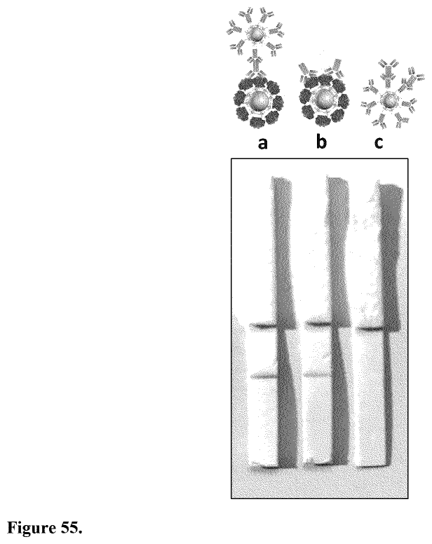

FIG. 55. (a) Picture of the strip incubated with Ag-C2-S1-pc and Ag-C2-Anti-Human-IgG-pc (OD=0.1) mixed with 200 ng/mL of Anti-Sars-Cov2 Human IgG. (b) Picture of the strip incubated with Ag-C2-S1-pc (OD=0.1) mixed with 200 ng/mL of Anti-Sars-Cov2 Human IgG. (c) Picture of the strip incubated with AgC2-Anti-Human-IgG-pc (OD=0.1) mixed with 200 ng/mL of Anti-Sars-Cov2 Human IgG.

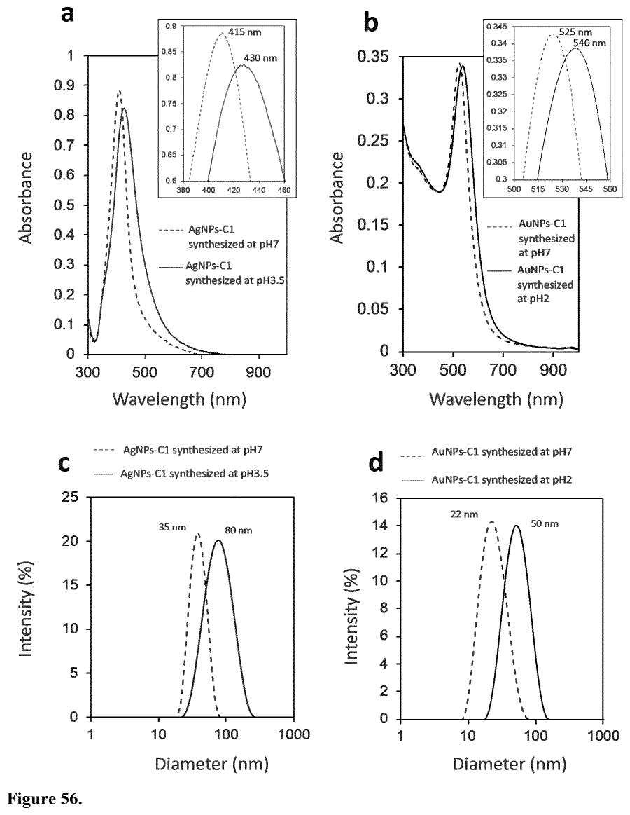

FIG. 56. (a) UV-Vis spectra of Ag—C1 synthesized at pH 7 (black dashed line) or pH 3.5 (blue straight line) dispersed in water. (b) UV-Vis spectra of AuNPs-C1 synthesized at pH 7 (black dashed line) or pH 2 (red straight line) dispersed in water. (c) Hydrodynamic diameter of Ag—C1 synthesized at pH 7 (black dashed line) or pH 3.5 (blue straight line) dispersed in water obtained by DLS measurements. (d) Hydrodynamic diameter of AuNPs-C1 synthesized at pH 7 (black dashed line) or pH 2 (red straight line) dispersed in water obtained by DLS measurements.

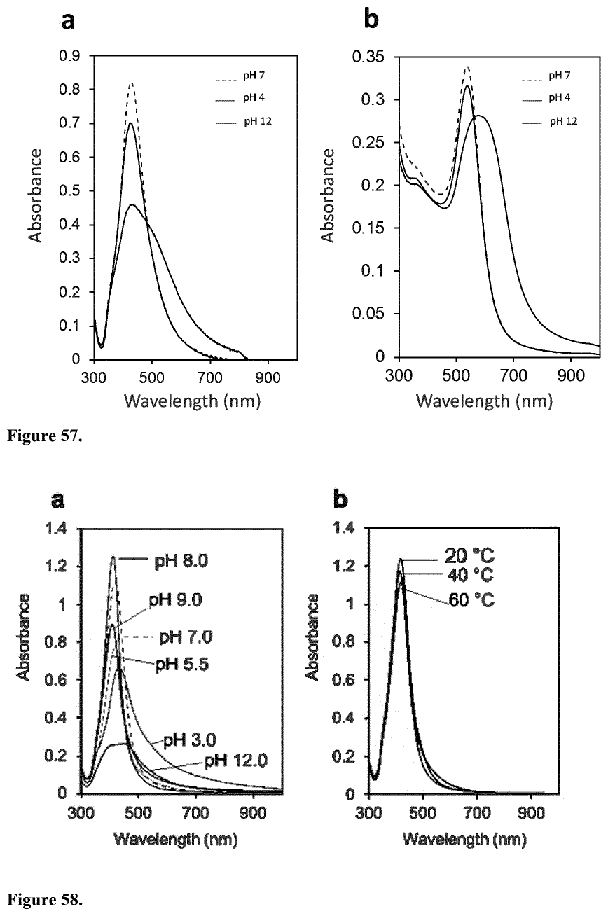

FIG. 57. (a) UV-Vis spectra of Ag—C1 synthesized at pH 3.5 dispersed in water at pH 7 then at pH 4 and finally at pH 12. (b) UV-Vis spectra of AuNPs-C1 synthesized at pH 2 dispersed in water at pH 7 then at pH 4 and finally at pH 12.

FIG. 58 (a) Absorbance spectrum of Ag—C1 synthesized at various pH; (b) Absorbance spectrum of Ag—C1 synthesized at various temperatures.

The term “about” when used in relation to a numerical value has the meaning generally known in the relevant art. In certain embodiments the term “about” may be left out or it may be interpreted to mean the numerical value+10%; or +5%; or +2%; or +1%.

The term “thickness” refers to the distance between the surface that is grafted and the part of the grafted (thia)calix[n]arene molecule that is furthest away from the surface. Usually an ultrathin layer of calixarene is grafted onto the metallic core. The ultrathin layer is preferably a monolayer. An ultrathin layer has a thickness of about 1 nm to 15 nm, corresponding to the calixarene rings without any further substituents (i.e. not counting R¹, R², R³, R⁴, R⁵ and R⁶ groups)

The terms “rather dense” and “dense” are used to describe a surface of a material that is coated with molecules in such way that molecules considered as single spheres or cylinders occupy an area equivalent to more than 50%, or more than 60%, or more than 70%, of a close-packed organization of the spheres or cylinders according to the compact van der Waals model.

The term “alkyl” refers to non-aromatic hydrocarbon groups. In particular “alkyl” refers to linear or branched, cyclic (e.g. cycloalkyl) and non-cyclic (acyclic) hydrocarbon groups. These may be unsaturated (see “alkenyl” and “alkynyl” below) or saturated. They can have varying numbers of carbon atoms, e.g. up to about 30, or up to about 20, or up to about 15, or up to about 10 carbon atoms. Alkyl groups thus include C₁₋₃₀ alkyl, C₁₋₁₀ alkyl (as more specifically defined below), C₁₋₆ alkyl, and C₁₋₄ alkyl groups.

The term “C₁₋₁₀ alkyl” denotes straight and branched saturated hydrocarbon radicals having from one to ten carbon atoms such as, for example, methyl, ethyl, 1-propyl, 2-propyl, 1-butyl, 2-butyl, 2-methylpropyl, 1-pentyl, 2-pentyl, 2-methylpropyl, 1-hexyl and other hexyl isomers, 1-heptyl and other heptyl isomers, 1-octyl and other octyl isomers, 1-nonyl and other nonyl isomers, 1-decyl and other decyl isomers. The term “C₁₋₆ alkyl” include one to six carbon atoms. “C₁₋₄alkyl” have from one to four carbon atoms. Of particular interest are straight (non-branched) C₁₋₁₀ alkyl, C₁₋₆ alkyl, or C₁₋₄ alkyl groups.

In the embodiments where two or more of R¹, R², R³, R⁴, R⁵ and R⁶ form a bridge (bridging group) selected from phosphine, phosphine oxide, amino, imino, amido, ureido, thioureido, ester, thioester, alkene, alkyne or alkyl, one of the hydrogen atoms or substituents on these moieties are replaced by a bond.

The calix[n]arene-based diazonium salts of formula I can be prepared by reacting a calix[n]arene bearing amino NH₂ groups with a nitrite such as sodium nitrite, in an aqueous acidic solution or with an alkyl nitrite such as isoamylnitrite or tertio-butylnitrite in an organic solvent (e.g. dichloromethane, polar aprotic solvents such as acetonitrile, dimethylformamide, dimethylacetamide, and the like solvents) or with nitroso salt (NOX) in organic solvents (e.g. acetonitrile). The diazonium salts of formula I, preferably the BF₄⁻ salts, can be prepared from an ice-cold solution of the corresponding amino NH₂ derivatives in HBF₄ by the slow addition of NaNO₂ (in excess) dissolved in a minimum amount of water. The precipitate is filtered off, washed with H₂O. The diazonium salts of formula I, preferably its BF₄⁻ salts, can be prepared from a solution of the corresponding amino NH₂ derivatives in acetonitrile in the presence of NOBF₄ (preferably in a slight molar excess) at low temperature (e.g. −40° C.). Typically, the crude residue is then washed with diethylether and/or ethanol.

The ultrathin layer of grafted (thia)calix[n]arenes is a homogeneous layer and does not present the large ramifications typically encountered with other systems, which lead to a complex and irregular surface coating. The grafted (thia)calix[n]arenes may form a rather dense coating, so that little free surface of the coated material is present.

Classical (bio)conjugation reactions such as peptide type coupling, maleimide-thiol reaction or copper catalyzed Huisgen cycloaddition (click chemistry) can be used for the immobilization of molecules or biomolecules on the grafted (thia)calix[n]arene ultrathin layer, the choice of the reaction depending on the groups present on the grafted (thia)calix[n]arenes. As a representative example, the grafted (thia)calix[n]arene ultrathin layer, when functionalized with a carboxylic acid group, can be esterified or converted with an appropriate amine into amides. Appropriate amines include not only simple amines but also amino acids, peptides, proteins, immunoglobulins, DNA, RNA, microRNA, and various chemical species (such as ligands for metal ions or for anions, molecular receptors, oligomers or polymers) with one or multiple appending amino arms. The carboxylic acid group can further be linked to hydroxyl-containing species such as saccharides, cyclodextrins and polyethylene glycols.

The covalent surface grafting of functionalized (thia)calix[n]arenes (with e.g. COOH, maleimide or alkyne groups on the small rim) on the large rim provides well-organized and compact monolayers, which can be post-functionalized. In other words, grafted (thia)calix[n]arenes induce a pre-structuration and a pre-functionalization of the surface at the molecular level.

The grafted materials of the invention can be used as a versatile platform for further modification, in particular the anchoring of further molecules resulting a regular and possibly rather dense molecular layer of various chemical species (molecules, nanoparticles, biomolecules, ligands for metal ions or anions, molecular receptors, oligomers or polymers, etc.) on conducting or semiconducting or non-conducting material surfaces. The method of the invention enables to control the density of functional groups present at the surface of the nanomaterials in order to introduce post-functionalization.

The calixarene layer can comprise various calixarenes.

The examples below make use of three types of calixarenes: a functional calixarene (C1), a functional PEGylated calixarene (C2) and a PEGylated calixarene (C3). The versatility of this procedure is even greater as the core can be made of metal alloys and the organic layer can be a mixed layer of calixarenes in order to combine their different properties.

The abbreviation NP or NPs designate nanoparticle(s).

The abbreviation LSPR refers to the Localized Plasmon Resonance band displayed by the metallic nanoparticles.

General Synthetic Scheme

Scheme 1 illustrates the general method of the invention to convert a mixture of at least one metal-based salt with at least one calix[4]arene diazonium salt (C₁, C₂ and/or C₃) to obtain a metal-based nanomaterial with a core being pure metal or an alloy, coated with a calixarene layer, of either single type or combined type.

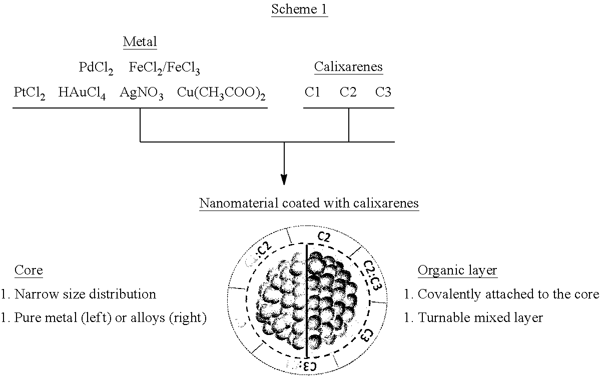

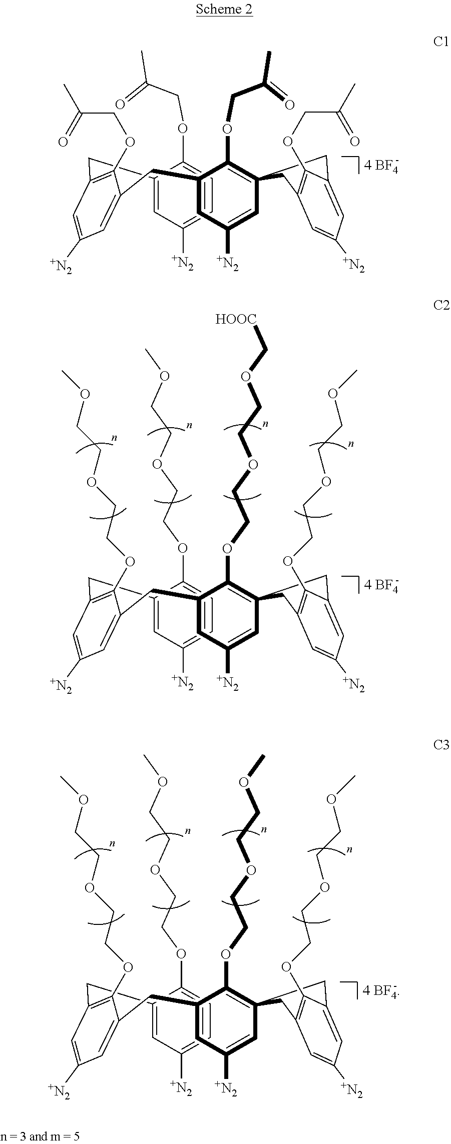

Scheme 2 shows the three calix[4]arenes that were used to make different layers, each possessing unique properties:
C1: this calixarene is negatively charged at pH values above 6, which ensures the stability of the colloids thanks to electrostatic repulsion between the particles. The carboxyl groups also allow post-functionalization of the particles with an amine or hydroxy-containing (bio)molecule via the formation of an amide or ester group using common coupling procedures. The particles coated with C1 are pH sensitive and precipitate at low pH. This aggregation should however be reversible, and the particles easily resuspended in basic conditions.
C2: this calixarene bears three PolyEthylene Glycol (PEG) chains ended by a methoxy group and one terminated by a carboxylic acid. This calixarene displays one protonable/deprotonable group, which should lead to particles that are only weakly pH sensitive. Furthermore, PEG is considered as the gold standard polymer to ensure biocompatibility and antifouling properties to nanomaterials. The presence of one carboxylic group allows post-functionalization of the particles.
C3: this calixarene bears four Polyethylene Glycol (PEG) chains ended by a methoxy group, which should lead to pH insensitive colloids.

Mixed layers of these calix[4]arenes possess combination of these properties with the possibility to provide to the functionalized particles the desired properties by controlling the proportion of the calix[4]arenes within the mixed layer.

General Synthesis Procedure 1.

In a protein LoBind eppendorf, 150 μL of a 10 mM metal salt solution are mixed successively with 575 μL of milliQ water and 360 μL of a solution of a 5 mM calixarene milliQ water and the pH is adjusted to 6.5 using a NaOH solution (1M). Quickly after this, 410 μL of a 15 mM sodium ascorbate solution are added and the Eppendorf is placed overnight in a thermomixer at 60° C. and agitated at 800 RPM.

The sodium ascorbate is present at around 4 mM concentration in the reaction mixture.

After 16 hours, the nanoparticles are centrifuged (20 minutes at 18.000 g) and the supernatant is removed and replaced by around 1500 μL of a washing medium. This washing procedure is repeated four times. The medium replacing the supernatant depends on the calixarene used (table 1). In the case of silver nanoparticles, the final concentration of nanoparticles obtained after the washing step is evaluated by measuring the extinction coefficient of the silver particles following the method disclosed in D. Paramelle, A. Sadovoy, S. Gorelik, P. Free, J. Hobley and D. G. Fernig, A rapid method to estimate the concentration of citrate capped silver nanoparticles from UV-visible light spectra, Analyst, 2014, 139, 4855.

TABLE 1
medium used to replace the removed supernatant
during the cleaning steps of nanoparticles.

| | C1 | C2 | C3 | C1:C2 | C1:C3 | C2:C3 |
|---|---|---|---|---|---|---|
| 1^{st} cycle | Water* | SDS** | SDS** | SDS** | SDS** | SDS** |
| 2^{nd} cycle | Water* | SDS** | SDS** | SDS** | SDS** | SDS** |
| 3^{rd} cycle | Water* | Water* | Water* | Water* | Water* | Water* |
| 4^{th} cycle | Water* | Water* | Water* | Water* | Water* | Water* |

*Lichrosolv water;
**1% SDS solution in weight

When the refractive index of a particle is known, the final concentration of particles can also be measured by DLS measurement (Malvern-Zetasizer) to give the concentration of particles (particles/mL).

Alternatively, the number of produced particles can be extrapolated from the quantity of reduced metal (the difference between the total quantity of metal added and the quantity of remaining cations in the solution, measured for example by ICP), the average size of the particles (obtained by TEM) and the density of the metal.

The UV-Visible spectrum of the coated nanomaterial is recorded using an absorption spectrometer. The coated nanomaterials are typically diluted 10 to 12 times in pure water for recording the UV-Visible spectra.

The colloidal stability is evaluated by comparing UV-Vis spectrum of the coated nanomaterial at pH 3, pH 7 and pH 12. Alteration of the UV-Visible spectrum at pH 3 is the sign of particles aggregation. Reversibility can be evaluated by assessing recovery of the UV-Vis spectrum when increasing the pH.

The chemical stability of the coated nanomaterial is evaluated by recording the UV-Visible spectra of the coated nanomaterial in solution at different time intervals after addition of a 150 mM potassium fluoride solution. No or little alteration of the UV-Visible spectrum means that the colloidal stability is not affected by the addition of KF, indicating that the metallic surface is not accessible to the fluoride ions, which is a sign of a dense and homogeneous repartition of the calixarenes on the surface of the metal-based core.

The presence of calixarenes on the metallic core is confirmed by IR spectroscopy.

The size and the shape of the metallic core of the particles was determined by analysis with SEM (scanning electron microscopy) and/or TEM (transmission electron microscopy).

The hydrodynamic diameter of the particles is analyzed by Dynamic Light Scattering (DLS) at a 100× dilution in water.

General observations for the experimental conditions:
a pH around 6.5 (±1) of the reaction medium leads to the best coverage of nanoparticles with calixarenes.
the core size can be tuned by changing the reductive strength of the medium in which the particles are synthesized. The nature and amount of the reducing agent in the general synthesis procedure (sodium ascorbate) leads to the formation of approximately 20 nm particles, which is the most commonly desired size for biomedical applications.

### Example 1: Silver Core Nanomaterials Coated with Calix[n]Arenes of Type C1: Ag—C1-20

Nanoparticles with pure silver core and coated with calixarene C1 were synthesized according to the general synthesis procedure 1, using silver nitrate as silver salt.
The UV-visible spectrum of Ag—C1-20 (FIG. 1) shows a sharp absorption peak at 418 nm, revealing well-dispersed silver nanoparticles with a narrow size distribution.
The final concentration of nanoparticles was estimated to be around 2.5 nM, based on a calculated extinction coefficient of 4.10⁹ L.mol^{-l}.cm⁻¹, which is in good agreement with the extinction coefficient reported in the literature for particles of that size (20 nm as shown by TEM).
Colloidal stability is affected at low pH the Ag—C1-20 particles are aggregated as the carboxylic acids of C1 are protonated, increasing the initially negative surface potential of the particles, and decreasing the electrostatic repulsion between them. A typical spectrum of DLCA (Diffusion Limited Colloidal Aggregation) aggregated particles with a second absorption band around 490 nm is observed. However, this aggregation is reversible by increasing the pH thanks to the highly protective calixarene layer.
Chemical stability—After 12 hours of exposure to potassium fluoride, only few particles were degraded, meaning that the silver surface is hardly accessible to the fluoride thanks to the dense and strongly anchored calixarene layer (FIG. 2).
The IR spectrum of Ag—C1-20 (FIG. 4) clearly shows absorption peaks of the aromatics (1458 cm-1) and of the carboxylates (1620, 1350, 1050 cm-1) of the calixarene. SEM and TEM showed spherical particles with a core size of around 20 nm (FIG. 5).
The hydrodynamic diameter of Ag—C1-20 diluted 100 times in milliQ water at 25° C. is around 35 nm
The synthesis was repeated at larger scales (1.5 mL, 20 mL, 30 mL of metal salt solution. A larger LSPR and a broader size distribution of articles was observed when increasing the scale to 20 mL and 30 mL. A further low speed centrifugation step (5000 g) was added at the end of the synthesis to remove the largest nanoparticles and narrow the LSPR.

### Example 1(b): Effect of the Temperature on Silver Core Nanomaterials Coated with Calix[n]Arenes of Type C1: Ag—C1-20

Nanoparticles with pure silver core and coated with calixarene C1 were synthesized according to the general synthesis procedure 1 and example 1, but at three different temperatures: 20° C., 40° C. and 60° C.
The UV-visible spectrum (FIG. 58(b)) of the particles obtained for the three reaction temperatures is the same.

### Example 1(b): Effect of the pH on Silver Core Nanomaterials Coated with calix[n]arenes of Type C1: Ag—C1-20

Nanoparticles with pure silver core and coated with calixarene C1 were synthesized according to the general synthesis procedure 1 and example 1, but at various pH ranging between 3.0 and 12.0.
The UV-visible spectrum (FIG. 58(a)) of the particles obtained show that particles were formed at all pH, even if the sharpest LSPR band are obtained for particles prepared at pH between 7 and 8.

### Comparative Example 1

The chemical stability of commercially available silver nanoparticles, coated with citrate (Ag-Citrate, S-20-XX, Cytodiagnostics (Burlington, Canada)) or by a thiolated-PEG ended by a carboxylic acid (Ag-SPEG, SC3K-20-X*, Cytodiagnostics (Burlington, Canada)) was recorded for comparison under the same conditions as Ag—C1.
As shown on FIG. 3, in presence of KF, Ag-Citrate is almost completely degraded after 10 min, while Ag-SPEG slowly degrades overtime, revealing the robustness of the calixarene coating.
In terms of shelf life, LSPR analysis of Ag—C1 showed no degradation after 29 months, while a small shift was observed after 4 months for Ag-citrate and almost full degradation was observed at 4 months for Ag-SPEG.

### Comparative Example 2

Nanoparticles with pure silver core and coated with calixarene C1 were synthesized according to the method disclosed in Troian-Gautier et al., Chemcomm, 2016, (used to prepare gold nanoparticles with the calix[4]arene C1 in acetonitrile at 0° C., through a reduction with NaBH₄), using either acetonitrile or water as solvent. The protocol for washing the particles was either the one of Troian Gautier or the protocol of General procedure 1. The observations are gathered in the table below:

| | Washing | |
|---|---|---|
| Solvent | procedure | Observations |
| Acetonitrile | Troian-Gautier | No isolable particles, particles degraded |
| Water | Troian-Gautier | No isolable particles, particles degraded |
| Acetonitrile | Gen. Proc. 1 | 10 times less particles than with |
| | | synthesis of General Procedure 1, broad |
| | | size distribution of particles |
| water | Gen. Proc. 1 | 10 times less particles than with |
| | | synthesis of General Procedure 1, broad |
| | | size distribution of particles |

This experiment demonstrates the superiority of the method of the invention to prepare coated nanomaterials.

### Example 2a, 2b, 2c: Size Variation of Silver Core Nanomaterials Coated with Calix[n]arenes of type C1

Nanoparticles with pure silver core and coated with calixarene C1 were synthesized according to the general synthesis procedure 1 using silver nitrate as silver salt, but using, instead of a final 4 mM sodium ascorbate concentration:
a) aqueous sodium borohydride (NaBH₄), at 4 mM, which is a stronger reducing agent than ascorbate
b) a sodium ascorbate at 4 mM, as in example 1,
c) sodium ascorbate at 1 mM.
   The UV-Visible spectra of the resulting nanoparticle suspensions are shown in FIG. 6. TEM pictures are shown in FIG. 7.
   Example 2a: the particles show an absorbance peak at 405 nm, indicating smaller particles were synthesized compared to example 1. This is confirmed by TEM analysis that reveals a core size of 6 nm for these particles.
   Example 2b: the particles show an absorbance peak at 418 nm, as in example 1. This is confirmed by TEM analysis that reveals a core size of 24 nm for these particles.
   Example 2c: the particles show an absorbance peak at 432 nm, indicating that larger particles were synthesized compared to example 1. This is confirmed by TEM analysis that reveals a core size of 42 nm for these particles.

### Example 2d: Size Variation of Silver Core Nanomaterials Coated with Calix[n]Arenes of Type C1

Nanoparticles with pure silver core and coated with calixarene C1 were synthesized according to the general synthesis procedure 1 using silver nitrate as silver salt, but pH is maintained at 3.5 (no addition of NaOH during the synthesis). The freshly synthesized silver nanoparticles were analyzed by UV-Vis spectroscopy. A strong and sharp LSPR band was observed with a maximum of absorbance at 430 nm, (FIG. 56 a and c). It represents a shift of the maximum of absorbance of approximately 15 nm compared to Ag—C1-20, indicating the formation of larger nanoparticles.
This was confirmed by DLS measurement that revealed a hydrodynamic diameter approximately 2.5 times bigger than the one of Ag—C1-20 prepared at pH7 (FIG. 56). From these results, we can conclude that the synthesis in acidic conditions (without pH adjustment) leads to the formation of larger nanoparticles with a size of approximately 45 nm (Ag—C1-45).
The colloidal stability of these particles was similar to the one of 20 nm particles (FIG. 57a), i.e. the particles aggregate in acidic conditions (pH4) but can be dispersed back in basic conditions (pH12).

### Example 3: Functionalization of Ag—C1

Calixarene C1 bears functional carboxylate groups, allowing additional chemical reactions (post-functionalization of the surface). Formation of an amide bond between carboxylates of Ag—C1 and ligands containing an amine group was demonstrated with NH₂-PEG₇-OCH₃ (FIG. 8, left) and a peptide, whose sequence is AAPLSQETFSDLWKLL (FIG. 8, right).
FIG. 8 displays the IR spectra of calixarene coated nanomaterial Ag—C1 post-functionalized with NH₂-PEG₇-OCH₃ (FIG. 8, left) or the above-mentioned peptide (FIG. 8, right). Peaks corresponding to an amide bond are clearly present around 1650 cm⁻¹.

### Example 4: Silver Core Nanomaterials Coated with Calix[n]Arenes of Type C2: Ag—C2

Nanoparticles with pure silver core and coated with calixarene C2 were synthesized according to the general synthesis procedure 1, using silver nitrate as silver salt.
The UV-visible spectrum of Ag—C2 (FIG. 10) shows a sharp absorption peak at 424 nm, revealing well-dispersed silver nanoparticles with a narrow size distribution.
The final concentration of nanoparticles was estimated to be around 0.4 nM, based on a calculated extinction coefficient of 2.10¹⁰ L.mol⁻¹.cm⁻¹, which is in good agreement with the extinction coefficient reported in the literature for particles of that size (35 nm as shown by TEM).
Colloidal stability and chemical stability are affected by pH variation: during a pH variation cycle (neutral-acidic-basic) and upon KF exposure a loss of Ag—C2 particles is observed, both indicating a less efficient coating than in the case of Ag—C1 calixarenes (FIG. 10, left). However, the resistance of these particles to fluoride etching (FIG. 10, right) is still much higher than that of commercially available particles (ordered from Cytodiagnostics, Burlington, Ontario, Canada; core size of 20 nm; coated with a HS-PEG-OCH₃ (Mw=3000 Da).
The IR spectrum of Ag—C2 (FIG. 11) clearly shows absorption peaks of the aromatics (1458 cm⁻¹) and of the polyethyleneglycol (PEG) chains (1100 cm⁻¹) of the calixarene.
SEM and TEM showed spherical particles with a core size of around 35 nm (FIG. 12).

### Example 5: Antimicrobial Properties of Ag—C1-20 and Ag—C2

In addition to interesting optical properties, the interest of silver cores lies in their antimicrobial activity. Despite the dense and stable coating of Ag—C1, these particles express similar or even greater antimicrobial activity than citrate-capped silver nanoparticles (Ag-Citrate).
E. Coli growth inhibition was studied by disc diffusion method for different conditions: 1=water; 2=Ag-citrate; 3=Ag—C1; 4=Ag—C2; 5=60 μM Kanamycine; 6=60 mM 30 Kanamycine).
FIG. 9 shows on the left part the test plate after 12 h, and on the right part, the optical density at 600 nm of bacteria suspension in presence of 1-water, 2=Ag-citrate, 3=Ag-C1-20 and 4=Ag—C2, after 12 hours.
From the test plate, it is clear that Ag—C1 and Ag—C2 are as efficient as Ag-Citrate in the case of the disc diffusion method and more efficient than 60 μM of Kanamycine, even if this well-known antibiotic is more than 1000 times more concentrated than the particles ([AgNPs]=0.6 nM and [Kanamycine]=60 μM).
In the case of the inhibition in solution, a stronger growth inhibition is observed with Ag—C1 than with Ag-Citrate, characterized by a lower optical density at 600 nm, after a defined incubation time, which could be explained by a longer lifetime of the Ag—C1 in the culture medium due to the protective calixarene-based layer. Ag—C2 is as efficient as Ag-Citrate.

### Example 6: Silver Core Nanomaterials Coated with Calix[n]Arenes of Type C3: Ag—C3

Nanoparticles with pure silver core and coated with calixarene C3 were synthesized according to the general synthesis procedure 1, using silver nitrate as silver salt.
The UV-visible spectrum of Ag—C3 (FIG. 13) shows a sharp absorption peak at 432 nm, revealing well-dispersed silver nanoparticles with a narrow size distribution.
The final concentration of nanoparticles was estimated to be around 0.4 nM, based on a calculated extinction coefficient of 2.10¹⁰ L.mol⁻¹.cm⁻¹, which is in good agreement with the extinction coefficient reported in the literature for particles of that size (around 46 nm as shown by TEM).
Colloidal stability and chemical stability are only slightly affected by pH variations (FIG. 13 left) and KF exposure (FIG. 13 right). This is due to the presence of the uncharged PEG layer capping the particles, which provide steric stabilization of the colloidal suspension instead of the electrostatic stabilization (which is the case for C1).
The IR spectrum of Ag—C3 (FIG. 14) clearly shows absorption peaks of the aromatics (1458 cm⁻¹) and of the polyethyleneglycol (PEG) chains (1100 cm⁻¹) of the calixarene.
SEM and TEM showed spherical particles with a core size of around 46 nm (FIG. 15).
pH is important for obtaining optimal nanoparticles. The optimal pH for a narrow size distribution of Ag—C3 is 7. Deviation from this pH leads to larger size distribution.

### Example 7: Silver Core Nanomaterials Coated with Calix[N]arenes of Type C1 and C2: Ag—C1C2

Nanoparticles with pure silver core and coated with a mixture of calixarenes C1 and C2, in various ratio, were synthesized according to the general synthesis procedure 1.
The results are summarized in table 1 below.

TABLE 1

| Molar fraction of C2 | 1 (Ag—C2) | 0.66 | 0.5 | 0.33 | 0 (Ag—C1-20) |
|---|---|---|---|---|---|
| UV absorption | 426 | | | | 418 |
| peak | | | | | |
| Colloidal | Idem | Minor | Minor | Reversible | Idem |
| stability | example 4 - | reversible | reversible | aggregation | example 1 - |
| | high | aggregation | aggregation | | reversible |
| | resistance | | | | aggregation |
| Chemical | | | No loss of | | |
| stability | | | particles | | |

The UV-visible spectra of the Ag—C1C2 (FIG. 16) show sharp absorption peaks between 426 and 418 nm, revealing well-dispersed silver nanoparticles with a narrow size distribution.
The final nanoparticle concentrations were estimated to be around 0.4-2.5 nM, based on the calculated extinction coefficient.
Colloidal stability and chemical stability: The chemical robustness of these hybrid Ag—C1C2 particles is demonstrated by exposing the particles to extreme conditions, either acidic (FIG. 16) or etching environment (FIG. 17). After a pH variation cycle, we observe high resistance of the hybrid particles against acidic conditions, without loss of particles, unlike in the case of pure Ag—C2. A mixed layer of calixarene C1:C2 increases the chemical robustness of these particles.
FIG. 17 shows the etching resistance for the Ag—C1C2 in a 1:1 ratio. These particles can endure fluoride exposure during several hours without significant loss.
The IR spectrum of Ag—C1C2 (FIG. 18) clearly shows absorption peaks of the aromatics (1458 cm⁻¹), the carboxylates (1620, 1350, 1050 cm⁻¹) and of the polyethyleneglycol (PEG) chains (1100 cm⁻¹) of the calixarene for all calixarene ratios, indicating that the calixarenes are all well grafted onto the surface. Furthermore, the intensity of the PEG signal depends on the amount of C2 added during the synthesis (the IR spectra have been normalized using the ring stretch band of the calixarenes at 1458 cm⁻¹), thereby confirming that it is possible to (i) synthesize particles covered with a mixed layer of calixarenes and (ii) control the proportions of calixarenes within the mixed layer.

### Example 8: Silver Core Nanomaterials Coated with Calix[n]arenes of Type C1 and C3: Ag—C1C3

Nanoparticles with pure silver core and coated with a mixture of calixarenes C1 and C3, in various ratio, were synthesized according to the general synthesis procedure 1.
The results are summarized in table 2 below.

TABLE 2

| | 1 | | | | | | | | | 0 |
|---|---|---|---|---|---|---|---|---|---|---|
| Molar fraction of C2 | (Ag—C3) | 0.95 | 0.90 | 0.85 | 0.80 | 0.70 | 0.60 | 0.5 | 0.35 | (Ag—C1-20) |
| UV peak (nm) | 432 | | | | | | | | | 418 |
| Colloidal | Idem | High | High | High | High | Minor | Minor | Minor | Reversible | Idem |
| stability | example 6 - | resistance | resistance | resistance | resistance | reversible | reversible | reversible | aggrega- | example 1 - |
| | high | to acidic | to acidic | to acidic | to acidic | aggrega- | aggrega- | aggrega- | tion | reversible |
| | resistance | conditions | conditions | conditions | conditions | tion | tion | tion | | aggregation |
| Chemical | | | | No loss of | | | | | | |
| stability | | | | particles | | | | | | |
| Core sire | 46 nm | | | | | 39 nm | | | | 24 nm |

The UV-visible spectra of the Ag—C1C3 (FIG. 19) show sharp absorption peaks between 432 and 418 nm, revealing well-dispersed silver nanoparticles with a narrow size distribution.
The final concentration of nanoparticles was estimated to be around 0.14-2.5 nM, based on the calculated extinction coefficient.
Colloidal stability and chemical stability: The chemical robustness of these hybrid Ag—C1C3 particles is demonstrated by exposing the particles to extreme conditions, either acidic (FIG. 19) or etching environment (FIG. 20). FIG. 20 shows the etching resistance for the Ag—C1C3 in a 15:85 ratio. These particles can endure fluoride exposure during several hours without significant loss.
The IR spectrum of Ag—C1C3 (FIG. 21) clearly shows absorption peaks of the aromatics (1458 cm-1), the carboxylates (1620, 1350, 1050 cm-1) and of the polyethyleneglycol (PEG) chains (1100 cm-1) of the calixarene. For all calixarene ratios, the calixarenes are all well grafted onto the surface. Furthermore, the intensity of the PEG signal depends on the amount of C3 added during the synthesis (spectra normalized as described in example 7), thereby confirming that it is possible to (i) synthesize particles covered with a mixed layer of calixarenes and (ii) control the proportions of calixarenes within the mixed layer.

### Example 9: Silver Core Nanomaterials Coated with Calix[n]arenes of Type C2 and C3: Ag—C2C3

Nanoparticles with a pure silver core and coated with a mixture of calixarenes C3 and C2, in various ratio, were synthesized according to the general synthesis procedure 1.
The results are summarized in table 3 below.
The UV-visible spectra of the Ag—C2C3 (FIG. 22) show sharp absorption peaks between 424 and 432 nm, revealing well-dispersed silver nanoparticles with a narrow size distribution.
The final concentration of nanoparticles was estimated to be around 0.1-0.4 nM, based on the calculated extinction coefficient, depending on the amount of calixarene C2.

TABLE 3

| Molar fraction of C2 | 1 (Ag—C2) | 0.85 | 0.66 | 0.5 | 0.33 | 0 (Ag—C3) |
|---|---|---|---|---|---|---|
| UV peak | 424 | | | | | 432 |
| (nm) | | | | | | |
| Colloidal | Idem | High | Mild | Reversible | Reversible | Idem |
| stability | example 4 - | resistance to | reversible | aggregation | aggregation | example 6 - |
| | | acidic | aggregation | | | high |
| | | condition | | | | resistance |
| Chemical | | No loss of | | | | |
| stability | | particles | | | | |
| Core sire | 46 nm | | | | | 24 nm |

Colloidal stability and chemical stability: The chemical robustness of these hybrid Ag—C2C3 particles is demonstrated by exposing the particles to extreme conditions, either acidic or etching environment (FIG. 22). FIG. 22 shows the etching resistance for the Ag—C2C3 in a 50:50 ratio. These particles are more resistant than commercially available particles.
The IR spectrum of Ag—C2C3 (FIG. 23) clearly shows absorption peaks of the aromatics (1458 cm⁻¹) and of the PEG chains (1100 cm⁻¹) of the calixarenes. For all calixarene ratios, the calixarenes are all well grafted onto the surface. Furthermore, the intensity of the PEG signal depends on the amount of C3 added during the synthesis (spectra normalized as described in example 7), thereby confirming that it is possible to (i) synthesize particles covered with a mixed layer of calixarenes and (ii) control the proportions of calixarenes within the mixed layer.

### Example 10: Gold Core Nanomaterials Coated with Calixarenes C1, C2 and/or C3

Several calixarene coated gold nanomaterials or gold and silver coated nanomaterials were prepared using the same experimental procedure but starting with a gold salt: HAuCl₄.
Data regarding UV and IR spectrum, stability studies are gathered in Table 4.
Overall, it was possible to product gold nanoparticles coated with a stable and homogeneous layer of calixarenes (single type or mixtures). These particles demonstrate high colloidal and chemical robustness and possess the capacity to be post-functionalized. Nanomaterials with a core comprising gold and silver were prepared, the ratio of metal incorporated in the core matching the ratio of the oxidized metal used for the synthesis. Similarly, to the silver nanomaterials described above, mixed layers of calixarenes could also be successfully be prepared, with a good control of the layer composition.

TABLE 4

| | | Au- | | |
|---|---|---|---|---|
| | | mercaptoundecanoic | | |
| | | acid | | |
| | Au-citrate | (Au-MUA) | Au—C1 | Au—C2 |
| calixarene | NA | NA | 100% C1 | 100% C2 |
| composition | | | | |
| detail of metallic | | | | |
| composition | | | | |
| Concentration | | | 6.5 nM | 3.2 nM |
| (nM) | | | | |
| extinction | | | 4 × 10⁸ | 8 × 10⁸ |
| coeficient | | | | |
| (L · mol⁻¹ · cm⁻¹) | | | | |
| absorption peak | | | 525 nm | 533 nm |
| Stability | | | | |
| acidic conditions | irreversible | reversible | reversible aggregation, | minor reversible |
| | aggragation | aggregation | absorption shifted to 590 nm | aggregation |
| basic conditions | | stable | stable | stable |
| KF | | degradation of | stable | stable |
| | | particles | | |
| figure UV | 24 | 24 | 24 | 26 |
| figure IR | | | 25 | 27 |
| presence of | | | aromatics | aromatics |
| calixarene on core | | | (1458 cm⁻¹) | (1458 cm⁻¹) |
| surface | | | carboxylates (1620, 1350, | PEG chains |
| | | | 1050 cm−1) | (1100 cm⁻¹) |
| Figure SEM | | | | |
| particule size | | | 19 nm | |
| (SEM) | | | | |
| particle shape | | | spherical | |
| post | | | NH2-PEG7-OCH3 (FIG. 25, | |
| fonctionalization | | | Au—C1-A) | |
| post | | | a peptide, whose sequence | |
| fonctionalization | | | is AAPLSQETFSDLWKLL | |
| | | | ((FIG. 25, Au—C1-B) | |

| | Au—C3 | Au—C1C2 | Au—C1C2 |
|---|---|---|---|
| calixarene | 100% C3 | 50% C1 | 66% C1 |
| composition | | | |
| detail of metallic | | | |
| composition | | | |
| Concentration | 3.2 nM | | |
| (nM) | | | |
| extinction | 8 × 10⁸ | | |
| coeficient | | | |
| (L · mol⁻¹ · cm⁻¹) | | | |
| absorption peak | 530 nm | | |
| Stability | | | |
| acidic conditions | minor reversible | minor reversible | minor reversible |
| | aggregation | aggregation | aggregation |
| basic conditions | stable | stable | stable |
| KF | stable | stable | stable |
| figure UV | 28 | 26 | 26 |
| figure IR | 30 | 27 | 27 |
| presence of | aromatics | aromatics | aromatics |
| calixarene on core | (1458 cm⁻¹) | (1458 cm⁻¹) | (1458 cm⁻¹) |
| surface | PEG chains | carboxylates (1620, 1350, | carboxylates (1620, 1350, |
| | (1100 cm−1) | 1050 cm⁻¹) | 1050 cm⁻¹) |
| | | PEG chains (1100 cm⁻¹) | PEG chains (1100 cm⁻¹) |
| Figure SEM | 36 | | |
| particule size | 22 nm | | |
| (SEM) | | | |
| particle shape | spherical | | |
| post | | | |
| fonctionalization | | | |
| post | | | |
| fonctionalization | | | |

| | Au—C2C3 | Au—C2C3 | Au—C2C3 | Au—C2C3 | Au—C1C3 | Au—C1C3 |
|---|---|---|---|---|---|---|
| calixarene | 85% C2 | 66% C2 | 50% C2 | 33% C2 | 90% C3 | 80% C3 |
| composition | | | | | | |
| detail of metallic | | | | | | |
| composiition | | | | | | |
| Concentration | 3.2 nM | 3.2 nM | 3.2 nM | 3.2 nM | | |
| (nM) | | | | | | |
| extinction | 8 × 10⁸ | 8 × 10⁸ | 8 × 10⁸ | 8 × 10⁸ | | |
| coeficient | | | | | | |
| (L · mol⁻¹ · cm⁻¹) | | | | | | |
| absorption peak | | | | | | |
| Stability | | | | | | |
| acidic | minor | minor | minor | minor | minor | minor |
| conditions | reversible | reversible | reversible | reversible | reversible | reversible |
| | aggregation | aggregation | aggregation | aggregation | aggregation | aggregation |
| basic conditions | stable | stable | stable | stable | stable | stable |
| KF | stable | stable | stable | stable | stable | stable |
| figure UV | 28 | 28 | 28 | 28 | 29 | 29 |
| figure IR | | | | | 30 | 30 |
| presence of | aromatics | aromatics | aromatics | aromatics | aromatics | aromatics |
| calixarene on | (1458 cm⁻¹) | (1458 cm⁻¹) | (1458 cm⁻¹) | (1458 cm⁻¹) | (1458 cm⁻¹) | (1458 cm⁻¹) |
| core surface | PEG chains | PEG chains | PEG chains | PEG chains | carboxylates | carboxylates |
| | (1100 cm⁻¹) | (1100 cm⁻¹) | (1100 cm⁻¹) | (1100 cm⁻¹) | (1620, 1350, | (1620, 1350, |
| | | | | | 1050 cm⁻¹) | 1050 cm⁻¹) |
| | | | | | PEG chains | PEG chains |
| | | | | | (1100 cm⁻¹) | (1100 cm⁻¹) |
| | | | | | proportional | proportional |
| particule size | | | | | | |
| (SEM) | | | | | | |
| particle shape | | | | | | |
| post | | | | | | |
| fonctionalization | | | | | | |

| | Au—C1C3 | Au—C1C3 | Au—C1C3 | Au—C1C3 |
|---|---|---|---|---|
| calixarene | 70% C3 | 60% C3 | 50% C3 | 35% C3 |
| composition | | | | |
| detail of metallic | | | | |
| composiition | | | | |
| Concentration | | | | |
| (nM) | | | | |
| extinction | | | | |
| coeficient | | | | |
| (L · mol⁻¹ · cm⁻¹) | | | | |
| absorption peak | | | | |
| Stability | | | | |
| acidic | minor | minor | minor | minor |
| conditions | reversible | reversible | reversible | reversible |
| | aggregation | aggregation | aggregation | aggregation |
| basic conditions | stable | stable | stable | stable |
| KF | stable | stable | stable | stable |
| figure UV | 29 | 29 | 29 | 29 |
| figure IR | 30 | 30 | 30 | 30 |
| presence of | aromatics | aromatics | aromatics | aromatics |
| calixarene on | (1458 cm⁻¹) | (1458 cm⁻¹) | (1458 cm⁻¹) | (1458 cm⁻¹) |
| core surface | carboxylates | carboxylates | carboxylates | carboxylates |
| | (1620, 1350, | (1620, 1350, | (1620, 1350, | (1620, 1350, |
| | 1050 cm⁻¹) | 1050 cm⁻¹) | 1050 cm⁻¹) | 1050 cm⁻¹) |
| | PEG chains | PEG chains | PEG chains | PEG chains |
| | (1100 cm⁻¹) | (1100 cm⁻¹) | (1100 cm⁻¹) | (1100 cm⁻¹) |
| | proportional | proportional | proportional | proportional |
| particule size | | | | |
| (SEM) | | | | |
| particle shape | | | | |
| post | | Cyanine dye with | | |
| fonctionalization | | terminal amino | | |
| | | group absorbing at | | |
| | | 800 nm (FIG. 31) | | |

| | Au—Ag—C1 | Au—Ag—C3 | Au—Ag—C3 | Au—Ag—C3 |
|---|---|---|---|---|
| calixarene | 100% C1 | 100% C3 | 100% C3 | 100% C3 |
| composition | | | | |
| detail of metallic | 50% Au-50% Ag | 66% Au-33% Ag | 33% Au—66% Ag | 0% Au—100% Ag |
| composition | (molar) | (molar) | (molar) | (molar) |
| Concentration | | | | 0.1 nM |
| (nM) | | | | |
| extinction | | | | |
| coeffcient | | | | |
| (L · mol⁻¹ · cm⁻¹) | | | | |
| absorption peak | 460 nm | ~510 nm | ~450 nm | 425 nm |
| Stability | | | | |
| acidic conditions | reversible aggregation, | | minor reversible | |
| | absorption shifted to | | aggregation | |
| | 590 nm | | | |
| basic conditions | stable | | stable | |
| KF | stable | | stable | |
| figure UV | 30 | 33 | 33 | 33 |
| figure IR | 34 | 34 | 34 | 34 |
| presence of | aromatics (1458 cm⁻¹) | aromatics (1458 cm⁻¹) | aromatics (1458 cm⁻¹) | aromatics (1458 cm⁻¹) |
| calixarene on core | carboxylates (1620, | PEG chains (1100 cm−1) | PEG chains (1100 cm−1) | PEG chains (1100 cm−1) |
| surface | 1350, 1050 cm−1) | | | |

### Example 10b: Size Variation of Gold Core Nanomaterials Coated with Calix[n]Arenes of Type C1

Nanoparticles with pure gold core and coated with calixarene C1 were synthesized according to the general synthesis procedure 1 using HAuCl₄ as gold salt, but pH is maintained at 2 (no addition of NaOH during the synthesis). The freshly synthesized gold nanoparticles were analyzed by UV-Vis spectroscopy. A strong and sharp LSPR band was observed with a maximum of absorbance at 540 nm, (FIG. 56 b and d). It represents a shift of the maximum of absorbance of approximately 15 nm compared to AuNP—C1, indicating the formation of larger nanoparticles.
This was confirmed by DLS measurement that revealed a hydrodynamic diameter approximately 2.5 times bigger than the one of Au—C1 prepared at pH7 (FIG. 56 b and d). From these results, we can conclude that the synthesis in acidic conditions (without pH adjustment) leads to the formation of larger nanoparticles with a size of approximately 45 nm (Au—C1-45).
The colloidal stability of these particles was similar to the one of 20 nm particles (FIG. 57b), i.e. the particles aggregate in acidic conditions (pH4) but can be redispersed in basic conditions (pH12).

### Example 11: Palladium, Platinum, Copper and or Iron Oxide Core Nanomaterials Coated with Calixarenes C1, C2 and/or C3

Several calixarene-coated palladium, platinum, copper oxide and iron oxide nanomaterials were prepared using general procedure 1, starting from the appropriate salts: palladium chloride, platinum chloride, copper acetate or mix of Iron Chloride salts (33% FeCl₂/66% FeCl₃)

### NB:

For gold or alloys involving gold, the metallic salts solutions have to be heated at 60° C. for 20 minutes before the addition of the calixarenes and ascorbate;
For iron nanoparticles, the sodium ascorbate solution is replaced by the same volume of a 2 M ammonia solution (NH₃ (aq)).
For Iron nanoparticles, the final concentration of Iron is around 10 mM instead of 1 mM described in the general procedure.
For copper nanoparticles, temperature higher than 70° C. and pH lower than 4.5 can lead to pure metallic copper core instead of copper oxide core.
For copper nanoparticles, the half of the water added is replaced by acetonitrile.
For copper nanoparticles, the ascorbate 15 mM added is replaced by a mixture composed of 50% of ascorbate 15 mM and 50% of NaBH₄ 150 mM.
For platinum nanoparticles, platinum salt (PtCl₂) is dissolved in DMSO then this solution (20 mM) is diluted 2 times with pure water.
For platinum nanoparticles, the calixarenes is added after the reductant, which are an equimolar mixture of ascorbate and NaBH₄.
   Analytical data regarding the coated nanomaterials are gathered in Table 5.

TABLE 5

| | Pd—C1 | Pd—C3 | Pt—C1 | Pt—C1-A | Cu—C1 | Fe—C1 |
|---|---|---|---|---|---|---|
| Variation to general | | | | Solvent: DMSO | | Reducing |
| procedure | | | | Reducing agent: | | agent: NaOH |
| | | | | NaBH4 | | |
| Absorption peak | | | | | 355 nm (emission | |
| | | | | | at 405 nm; | |
| | | | | | FIG. 41) | |
| Stability | | | | | | |
| Acidic condition | Reversible | | | | Reversible | |
| | aggregation | | | | aggregation | |
| Basic condition | Stable | | | | Stable | |
| KF | Stable | | | | Stable | |
| Figure UV | | | | Same UV profile | 40 | |
| | | | | as Pt—C1 | | |
| Figure IR | 35 | 37 | 39 | | 42 | 44 |
| Presence of | Aromatics | Aromatics | Aromatics | | Aromatics | Aromatics |
| calixarene on core | (1458 cm⁻¹) | (1458 cm⁻¹) | (1458 cm⁻¹) | | (1458 cm⁻¹) | (1458 cm⁻¹) |
| surface | Carboxylates | PEG chains | Carboxylates | | Carboxylates | Carboxylates |
| | (1620, 1350, | (1100 cm⁻¹) | (1620, 1350, | | (1620, 1350, | (1620, 1350 |
| | 1050 cm⁻¹) | | 1050 cm⁻¹) | | 1050 cm⁻¹) | cm⁻¹) |
| Particle size (SEM) | 45 nm | 80 nm | 40 nm | | 100 nm | 20 nm |
| Particle shape | Spherical | Spherical | Spherical | | Cubic shape | Spherical |
| | (FIG. 36) | (FIG. 38) | | | (FIG. 43) | (FIG. 45) |

These experiments show the versatility of the method of the invention, to make metallic cores coated with a monolayer of calixarenes. The metallic cores can be single metal, metal oxide or an alloy or metallic mixture. The ratio of the metals in the nanoparticle obtained is proportional to the ratio of oxidized metal introduced in the reaction mixture.
Depending on the metal, different shapes of nanoparticles can be obtained, in particular, nanospheres and nanocubes have been obtained.
The method of the invention works in water and in other solvents, like DMSO.
The method enables to obtain nanoparticles coated with several types of calixarenes, which is particularly convenient for introducing a variety of functionalities or post-functionalization possibilities. This can lead to advantageous properties in many different fields of activities, like microelectronics, ND, biomedical applications, catalysis, nanoreactors formed by calixarenes at the surface of particles, . . . .
The repeatability of the experiments, and stability of the nanoparticles enable to extrapolate the results of the above experiments to all types of calixarenes, and many more metals.

### Metal-Based Nanomaterials of the Invention for Use in Immunoassays.

Silver based and gold based nanomaterials coated with calixarenes according to the method of the invention were used for preparing various immunoassays, in particular Ag—C1-20 as prepared in example 1, Ag—C2 as prepared in example 4, as well as Au—C1 and Au—C2 as prepared in example 10.
Unless otherwise stated, in the examples below, the anti-SARS-CoV-2 human IgG, and S-protein relate to Anti-Spike-RBD fully human mAb(IgG) and Recombinant SARS-CoV-2, S1 Subunit Protein (RBD) ordered from Tebu-Bio and Ray-Biotech respectively. An anti-SARS-CoV-2 human IgG and IgM mix was also ordered from MyBiosource.

### Example 12—Adsorption of Anti-Human-IgG or IgM on Ag—C1-20 and Au—C1

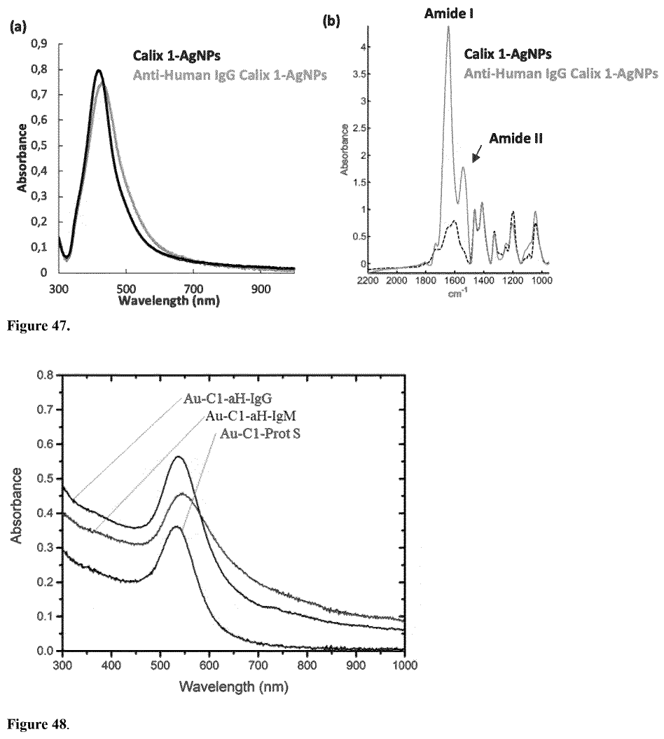

Ag—C1-20 or Au—C1 was stirred in the presence of 100 eq./NP of Human Anti-IgG (Goat Anti-Human IgG (I2136) acquired from Sigma-Aldrich (Saint-Louis, Mo.)) or Human Anti-IgM (Goat Anti-Human IgM acquired from Sigma-Aldrich (Saint-Louis, Mo.)) (both 2 mg/mL in PBS 10 mM ) during 2 h at room temperature at 1000 rpm to provide Ag-C1-Anti-Human-IgG-ad and Ag-C1-Anti-Human-IgM-ad particles respectively. After 2 hours, the mixture was centrifuged at 18000 g for 20 minutes. The LSPR had shifted from 417 nm to 426 nm, indicating a change in dielectric environment. The supernatant was discarded, and the pellet was resuspended in ultrapure water. This operation was repeated three times in total. The resulting particles were further characterized by IR spectroscopy. The presence of intense amide I and II bands confirmed the adsorption of anti-Human-IgG on the silver nanoparticles (FIG. 47).
Similarly, the shifts in the UV-visible spectra of the colloids confirmed the presence of Anti-human-IgG and Anti-human-IgM on the gold nanoparticles (FIG. 48).

### Example 13—Adsorption of Protein S

SARS-CoV-2 S protein was adsorbed onto calixarene coated silver particles with the aim to detect anti-SARS-CoV-2 human IgG in the blood of patients.
Protein-S was adsorbed on Ag—C1 based on adapted protocol found in literature. (ORCID iD: 0000-0001-8854-275X)
Three conditions were tested:
300 eq./NPs in pure water
300 eq./NPs in 10 mM borate buffer
100 eq./NPs in 10 mM borate buffer
   The UV-Vis spectra of the particles obtained in all conditions as well as the IR spectra are given on FIG. 49.
   In all three conditions, the adsorption of Protein S was confirmed by a red shift of the LSPR (from 417 nm to 424-427 nm) as well as by IR spectroscopy where intense signals characteristic of amide I and II bands were observed (FIG. 49).
   Adsorption of protein-S was also performed on Ag—C1 (FIG. 49), Au-citrate and Ag-citrate. In this case, a blocking buffer containing BSA (1 mg/ml BSA in 10 mM PBS) or Casein (0.1% Casein in 10 mM PBS) was added after 2 hours of adsorption and the resulting particles were cleaned in the same buffer and resuspended in pure water. Corresponding particles are named Ag-C1-S1-ad-BSA and Ag-C1-S1-ad-Casein, Au-citrate-S1-ad-BSA and Au-citrate-S1-ad-Casein or, Ag-citrate-S1-ad-BSA and Ag-citrate-S1-ad-Casein.

### Example 14—Bioconjugation of Anti-Rabbit IgG by Peptide Coupling

On the contrary to adsorption, bioconjugation involves creating at least one amide bond between a substituent of the calixarene coated to the surface of the metal nanomaterial and the protein (Immunoglobulin).
Attempts to bioconjugate anti-Rabbit IgG on silver nanoparticles stabilized with citrate, a thioalted PEG ended by a carboxylic acid (Ag-SPEG) or calix[4]arene were performed by activation of the carboxylic acid moieties with EDC/NHS. As can be seen from FIG. 50 a) and b), EDC/NHS coupling conditions are incompatible with Ag-citrate and Ag-SPEG. On the contrary, Ag—C1 were compatible for the bioconjugation reaction using anti-Rabbit IgG and EDC/NHS, yielding the corresponding Ag-C1-anti-rabbit-IgG-pc.
The bioconjugation is performed as follows:
In a glass vial, 250 μL of Ag—C1-20 (final OD=1), 380 μL of ultrapure water, 200 μL of PBS (pH 7.4, final C=20 mM), 100 μL of Sulfo-NHS (final C=0.1 mM), 100 μL of EDC (final C=0.06 mM) and 20 μL of rabbit anti-IgG (0.2 mg/mL) were added. The suspension was then stirred at room temperature at 1000 rpm for 4 hours. After 4 hours, 0.5 mL of BSA 1% in PBS 20 mM was added to the suspension and centrifuged at 18000 g for 20 minutes. The supernatant was discarded, and the pellet was resuspended in 1.5 mL of bovine serum albumin (BSA) 1% in PBS 20 mM. This operation was repeated once more to yield Ag-C1-anti-Rabbit-IgG-pc.
These nanoparticles were characterized by UV-Vis, DLS and IR spectroscopies. DLS confirmed the increase in hydrodynamic diameter upon bioconjugation. Indeed, this diameter increased from 37 nm for Ag—C1 to 180 nm for Ag-C1-anti-rabbit-IgG-pc. Infrared spectroscopy allowed to highlight the presence of amide bands I and II of the anti-rabbit IgG after bioconjugation.
Similarly, Bioconjugation of anti-Rabbit-IgG on Au—C1 was performed using the same procedure to yield Au-C1-anti-Rabbit-IgG-pc.

### Example 15—Bioconjugation of Anti-Human IgG by Peptide Coupling

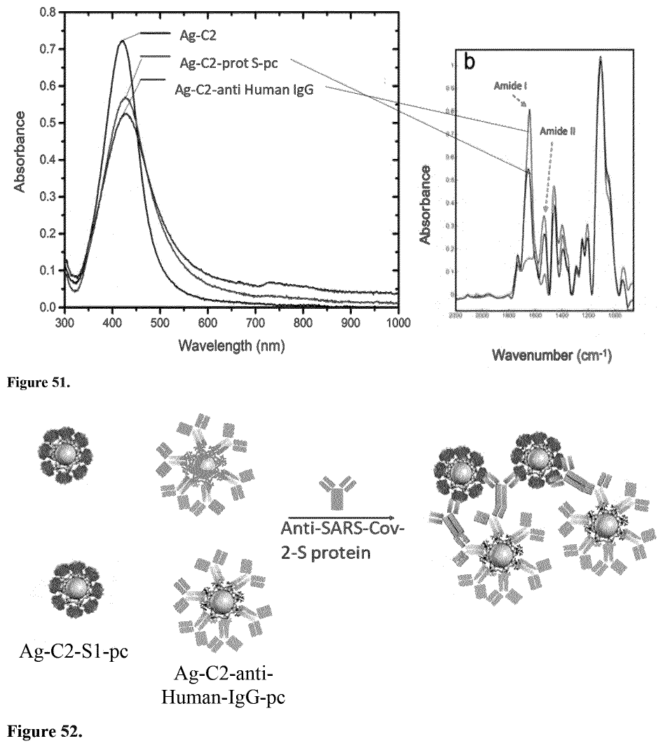

Bioconjugation of anti-human-IgG was performed on Ag—C1-20 and Au—C1. These reactions were carried out either in water or in borate buffer. Overall, it seems that carrying the reaction in borate buffer allows to obtain better results and to lose less nanoparticles.
To 1 mL of Ag—C1-20 (concentration=0.5 nM) or Au—C1 (concentration=5 nM) dispersed in ultrapure water in a glass vial were added 100 μL sulfo-NHS (final C=1 mM), 100 μL of EDC, and 50 μL of anti-human-IgG (2 mg/mL, approximately 100 eq./NP). The reaction was stirred at room temperature at 1000 rpm for 16 hours. After reaction, the mixture was split in two series. Series A was washed three times with ultrapure water and series B was washed once with SDS 1% and twice with water. Each time, the mixture was centrifuged at 18000 g for 20 minutes. This procedure yielded either Ag-C1-anti-human-IgG-pc or Au-C1-anti-human-IgG-pc. No relevant difference was observed for the final products obtained with washing of Series A and series B.
Anti-Human-IgG were also bioconjugated to Ag—C2. The nanoparticles were activated via the EDC/NHS procedure and cleaned from excess reagent via centrifugation cycles. These activated nanoparticles were then reacted overnight with approximately 100 biomolecules per nanoparticle. The corresponding Ag-C2-Anti-Human-IgG-pc were purified via centrifugation cycles and resuspended in PBS 10 mM and stored in the fridge. Bioconjugation was confirmed by UV-Vis and IR spectroscopy (FIG. 51). A shift of the LSPR absorption band was observed for the nanoparticles after bioconjugation. In addition, amide bands I and II are clearly observable after bioconjugation.

### Example 16—Bioconjugation of Anti-Human IgM by peptide coupling

Bioconjugation of anti-human-IgM was performed on Ag—C1-20 and Au—C1 as described above to yield Ag-C1-anti-human-IgM-pc and Au-C1-anti-human-IgM-pc. These reactions were carried out either in water or in borate buffer, as for example 16. Overall, it seems that carrying the reaction in borate buffer allows to obtain better results and to lose less nanoparticles. The obtained bioconjugated nanoparticles were characterized by UV-Vis spectroscopy as well as by IR spectroscopy. Amide bands I (1642 cm⁻¹) and II (1527 cm⁻¹) are clearly observable after bioconjugation.

### Example 17—Bioconjugation of SARS-CoV-2 S-Protein by Peptide Coupling

Bioconjugation of the S1 subunit of the SARS-CoV-2 S-Protein was performed on Au—C1 as in examples 16 and 17. These bioconjugated nanoparticles were characterized by UV-Vis spectroscopy as well as by IR spectroscopy. Amide bands I (1652 cm⁻¹) and II (1531 cm⁻¹) are clearly observable after bioconjugation. This procedure yielded Au-C1-S1-pc.
Bioconjugation of SARS-CoV-2 S-Protein was also performed on Ag—C1 as in examples 16 and 17. This procedure yielded Ag-C1-S1-pc. Additional treatment of the particles was obtained using a blocking protein, casein, added to the Ag-C1-S1-pc particles. After incubation with casein, particles were cleaned by centrifugation to yield Ag-C1-S1-pc-casein particles.
Coupling of Protein S was also performed of Ag—C2. The carboxylates were activated in MES buffer via the EDC/NHS procedure and cleaned from excess reagent via centrifugation cycles. These activated nanoparticles were then reacted overnight with approximately 100 biomolecules per nanoparticle. The corresponding Ag-C2-S1-pc were purified via centrifugation cycles and resuspended in PBS 10 mM and stored in fridge. Bioconjugation was confirmed by UV-Vis and IR spectroscopy.

### Example 18—Detection assay for Rabbit-IgG: Comparison of AgNPs and AuNPs

Rabbit-IgG solutions (1 mg/mL in PBS 10 mM, approximately 100 eq./NP; Rabbit IgG (I5006) acquired from Sigma-Aldrich (Saint-Louis, Mo.)), were added to solutions of Ag—C1-20 (0.24 nM) or Au—C1 (2.4 nM) suspended in PBS 50 mM at pH 7.4 to reach IgG concentrations varying from 0 to 220 nM. An UV-vis spectrum of each mixture was recorded. The ratio between the absorbance of aggregated nanoparticles ((λₘₐₓ+175 nm) and the absorbance of dispersed nanoparticles (λmax) was reported to estimate the aggregation level.
In these conditions, Ag—C1-20 allowed to detect Rabbit-IgG concentrations as low as 1.6 nM while Au—C1 are unable to detect Rabbit IgG at concentrations lower than 20 nM (see FIG. 46). Hence, silver nanoparticles allow to design more efficient molecular sensing system with a higher sensitivity, lower detection limit and lower concentration than the corresponding gold analogues.
The adsorption (aggregation) process was shown to be reversible upon washing with sodium dodecyl sulfate (SDS).

### Example 19—Detection Assay for COVID-19: Immunoturbidimetry

Molecular sensing of human antibodies was performed. The analyte was a Human IgG capable of binding specifically the S1 subunit of the SARS-CoV-2 S-protein. Two batches of silver nanoparticles were used. One was bioconjugated with the subunit S1 of the SARS-CoV-2 S-protein (Ag—C2-S1-pc). The second batch was bioconjugated with an Anti-Human IgG, i.e. Ag-C2-Anti-Human-IgG-pc, capable of recognizing specifically Human IgGs. In the presence of anti-Sars-CoV-2 human IgG (Anti-Spike-RBD fully human IgG), the particles aggregate via the formation of a sandwich-type ternary complex, leading to a modification of their optical properties and thus the detection signal. A scheme of the procedure is represented in FIG. 52.
The two sets of bioconjugated particles, i.e. Ag-C2-S1-pc and Ag-C2-Anti-Human-IgG-pc were mixed together in a quartz cuvette with 5 mM of PBS. Specific volumes of antibody solution were then added, and the UV-Vis spectrum was recorded after 10 minutes of incubation. IgG anti-SARS-CoV-2 (i.e. Anti-Spike-RBD fully human mAb(IgG) ). As a control, Rabbit-IgG was use. A linear increase of the absorbance at 600 nm, signature of aggregation of the particles, was observed when adding 1 nM to 4 nM of fully Human anti-SARS-CoV-2, but not with addition of Rabbit-IgG, even at concentration up to 250 nM.
In conclusion, this detection system allowed the specific detection of anti-Sars-Cov2 IgG with a low limit of detection at 1 nM.
Comparative experiments were run using Ag-citrate and Au-citrate nanomaterials, giving a much less sharp detection result.
Similar experiments were performed with Ag-C1-S1-pc and Ag-C1-Anti-Human IgG-pc, onto which anti-SARS-CoV-2 human IgG was added at increasing concentration (2 nM to 23.2 nM), giving a linear decrease and broadening of the LSPR.
These assays demonstrate the feasibility of a turbidimetry to detect immunoglobulins, and in particular anti-SARS-CoV-2 human IgG. These are very promising results towards the preparation of an immunoassay based on nanomaterials coated with calixarenes.

### Example 20—Vertical Flow Assay for SARS-Cov-2 Detection

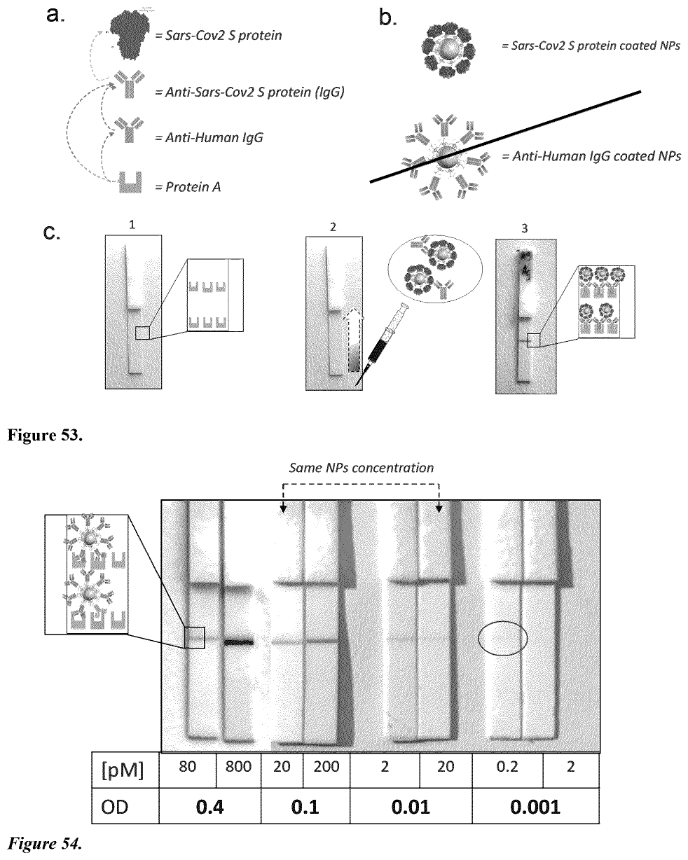

The examples below relate to the development of Lateral Flow Immunoassays (LFIA). In such an assay, the objective is to immobilize the functionalized nanoparticles on a strip in the presence of the analyte to detect, leading to the formation of a colored band that can be detected by the naked eye or a suitable detection technique. The sample to analyze is deposited on one extremity of the strip, combined with the functionalized nanoparticles and move towards the detection band by capillarity. If the analyte to be detected is present, it forms a bridge between the functionalized nanoparticles and the proteins/immunoglobins used to functionalize the strip at the level of the detection band, leading to the immobilization of the particles on this band and its coloration in red or yellow, depending on the use of AuNPs or AgNPs.
For development purpose, the assay is set up vertically, meaning that instead of depositing the analyte solution on an extremity of the strip, the extremity of the strip is plunged vertically in the analyte solution which moves up by capillarity along the strip.
Detection of anti-SARS-CoV-2 human IgGs was performed via a vertical flow assay involving strips functionalized at the level of the detection band with the protein A. The protein A is able to specifically bind to the heavy chains of IgG. The strips coated with proteins A and G were ordered from Abcam (Cambridge, United-Kingdom) as part of “Conjugation Check&Go” kit (ab236554).
In this context, anti-SARS-CoV-2 human IgGs can be immobilized on the detection band, and this interaction can be visually highlighted with engineered plasmonic nanoparticles (FIG. 53 a)).

1. Comparison Between Gold and Silver Nanoparticles

Calixarene-coated silver nanoparticles (Ag—C1-20) conjugated to Anti-Human IgG via adsorption (Ag-C1-Anti-Human-IgG-ad), as prepared in example 13, were compared to citrate-capped gold nanoparticles (Au-Citrate) conjugated to Anti-Human IgG via adsorption (Au-Citrate-Anti-Human-IgG_ad). In both cases, the particles can be immobilized on the strip via the interaction between the protein A and the Anti-Human IgG (FIG. 53).
Control experiments were first performed to show the effect of the bioconjugation. It was confirmed that, in the absence of conjugation, Ag—C1 does not get immobilized on the strip.
Nb: All the Ag-C1-biomolecules used in this part come from the NPs batches cleaned with Casein and described above in “Nanoparticles Bioconjugation” part.
Strips, onto which a band of protein A and G was present, were vertically plunged into 100 μL of either Ag-C1-Anti-Human-IgG-ad-Casein suspensions, at concentrations ranging from 0.2 to 80 pM, or Au-Citrate-Anti-Human-IgG-ad-Casein suspensions, at concentrations ranging from 2 to 800 pM. After 20 minutes of incubation/migration the strips were removed from the suspensions and pictures were taken (FIG. 54).
Ag-C1-Anti-Human-IgG-ad-Casein could be clearly observed on the strip for concentrations as low as 2 pM, and even detected at extremely low concentration (0.2 pM), with the naked eye. For Au-Citrate-Anti-Human-IgG-ad-Casein, concentration of 20 pM is the limit of observable signal with the naked eye and no coloration of the detection band could be observed at lower concentrations.
Ag-C1-Anti-Human-IgG-ad-Casein at the same concentration (20 pM) than gold nanoparticles lead to a much stronger signal thanks to their higher extinction coefficient.

2. Anti-Sars-Cov2 Human IgG Detection in Buffer

Ag—C1 conjugated with the SARS-CoV-2 S-protein via adsorption (Ag-C1-S1-ad-Casein) was used for the detection of anti-SARS-CoV-2 human IgG. Briefly, the strips, onto which a band of protein A and G has been deposited, were vertically incubated with 100 μL of solutions containing different concentrations of anti-SARS-CoV-2 human IgG (From 500 ng/mL to 1 ng/mL initially in PBS buffer) mixed with Ag-C1-S1-ad-Casein (initially in pure water) in buffer provided by the strip supplier, at an optical density of 0.1.
Strong detection signals could be observed down to concentration of 20 ng/mL of anti-SARS-CoV-2 human IgG. Concentration of 5 ng/mL could barely be observed by the naked eye and concentration of 1 ng/mL led to absolutely no signal. Control experiments confirmed the specificity of the interaction: (i) in the absence of anti-SARS-CoV-2 human IgG, no signal was observed and (ii) in the presence of Rabbit-IgG, no signal was observed.
In order to enhance the detection signal for concentration of 5 ng/mL, a higher concentration of silver nanoparticles was used (OD=0.4). The signal obtained was stronger than the one obtained with an OD of 0.1 Optimizing the concentration of nanoparticles appears to help to obtain a detectable signal at low IgG concentration.
Another strategy investigated was to use Ag—C1 conjugated to the viral S-protein via peptide coupling (Ag-C1-S1-pc-Casein). No significant difference was observed with this conjugation strategy compared with bioconjugation by adsorption

3. Anti-Sars-Cov2 Human IgG Detection in Pure Buffer with Ag—C2

Ag—C2 conjugated with the SARS-CoV-2 S-protein (Ag-C2-S1-pc) and Anti-human IgG (Ag-C2-Anti-Human-IgG-pc), both via amide bond formation, were used for the detection of anti-SARS-CoV-2 human IgG. Strips were vertically incubated with 100 μL of solution containing 200 ng/mL of anti-SARS-CoV-2 human IgG mixed with either Ag-C2-S1-pc, Ag-C2-Anti-Human-IgG-pc or both at an optical density of 0.1.
No signal was observed for the Ag-C2-Anti-Human-IgG-pc only, probably due to the saturation of the particles by the different IgGs present in the sample, preventing any interaction with the strip. In the other hand, Ag-C2-S1-pc allowed the visual detection of anti-SARS-CoV-2 human IgG.
An even stronger signal was obtained with the sandwich strategy involving the simultaneous presence of the two bioconjugated nanoparticles. This may be explained by the immobilization of nanoparticle aggregates on the strip instead of single particles (FIG. 55).
The above examples demonstrate that AgNPs coated with calixarenes and conjugated with biomolecules lead to more intense signal than gold nanoparticles when used in similar conditions, due to their higher extinction coefficient: signal detection can be obtained with very low concentration of silver NPs (down to 2 pM), i.e. more than ten times lower than their gold counterparts. Calixarene-coated silver nanoparticles can be used for the specific anti-SARS-CoV-2 human IgG detection both in immunoturbidimetry and lateral flow immunoassays. LFIAs using AgNPs only requires very low amount of material (down to 2 pM of AgNPs); as a comparison, UV-Vis spectroscopy requires typically 80 pM of silver NPs.

### Example 21—Vertical Flow Assay for SARS-CoV-2 Detection in Human Serum

The signal intensity with serum containing different amount of anti-SARS-CoV-2 human IgG: 5, 2.5, 1.5, 1, 0 μg/mL were compared.
10 μL of serum (containing different concentrations of IgG), 10 μL of Ag-C1-S-Prot-Casein-ad (OD=5), 5 μL of Rabbit IgG-AuNPs (provided by Abcam) for control line and an appropriate amount of running buffer to reach a total volume of 50 μL were used to dip therein strips onto which a band of Protein A is present.
A signal can be easily read on the strip for serum containing 5 and 2.5 μg/mL of SARS-Cov-2 IgG. For serum containing 1.5 μg/mL, a poorly discernible signal can also be observed. No signal can be observed for serum containing 1 and 0 μg/mL.

### Example 22—Vertical Flow Assay for SARS-Cov-2: Influence of the Type of Nanoparticles on the Detection Limit

3 different batches of nanoparticles with different coatings and bioconjugation strategies were compared: one type where the proteins are adsorbed onto the calixarene C1 coated nanoparticles Ag-C1-S1-ad-Casein, and two types where the proteins are bioconjugated by peptide coupling to the calixarene coated nanoparticles of Ag-C1-S1-pc-Casein and Ag-C2-S1-pc. The volumes of NPs solutions were adjusted in order to have similar OD in the different assay suspensions.
All NPs were tested with serum containing 2.5, 1.5, 1.0 and 0 μg/mL of anti-SARS-CoV-2 human IgG. Overall, peptide coupled NPs (pc) allowed to improve the limit of detection, giving a reliable signal at 1.5 μg/mL and weak signal at 1.0 μg/mL.
These results confirm the better bioconjugation through amide coupling compared to the mere chemical adsorption.

### Example 23—Vertical Flow Assay for SARS-Cov-2 with Two Simultaneous Types of Immunoglobulins

It was tested to deposit, on a same strip, two parallel lines, one composed of anti-human IgG and one composed of anti-human IgM. When a sample containing a mix of both the anti-SARS-CoV-2 human IgG and IgM incubated with Ag-C1-S1-pc-Casein in the presence of 10 μL serum (containing 1.5 μg/mL IgG and IgM mix) was used, both bands became visible.

1. A method to synthesize metal-based nanomaterials coated with calix[n]arenes comprising
placing at least one oxidized metal with at least one calix[n]arene diazonium salt in the presence of a reducing agent in a solvent, and
heating the reaction mixture to obtain a metal-based nanomaterial coated with calix[n]arenes.

2. The method according to claim 1, further comprising adjusting the pH of the reaction mixture at a value between 3.5 and 10.

3. The method according claim 1, wherein the molar ratio of oxidized metal to the calix[n]arene diazonium salt is between 10:1 and 1:10.

4. The method according to claim 1, wherein the oxidized metal comprises a metal oxide and/or a metal halide, nitrate, sulfate, carboxylate, triflate, sulfonate, phosphate, tosylate or a borate.

5. The method according to claim 1, wherein the metal of the oxidized metal is an alkali metal, an alkaline earth metal, a lanthanide, an actinide, a transition metal, a post transition metal, and/or a metalloid.

6. The method according to claim 5, wherein the metal is selected from the group consisting of silver, palladium, gold, platinum, copper, nickel, zinc, cadmium, indium, lead, titanium, tantalum, silicon, aluminum, and iron.

7. The method Hatred-according to claim 1, wherein the calix[n]arene diazonium salt is a calix[4]arene diazonium salt.

8. The method according to claim 1 wherein the reducing agent is a hydride or an ascorbate salt.

9. The method according to claim 1, wherein the solvent is water.

10. The method according to claim 1, wherein the reaction mixture is heated between 15° C. and 150° C.

11. The method according to claim 1, further comprising a step of bioconjugation of the calix[n]arenes with a protein.

12. The method according to claim 11, wherein bioconjugation is done using passive adsorption, peptide coupling or copper(I)-catalyzed alkyne-azide cycloaddition (CuAAC).

13. A metal-based nanomaterial comprising a metallic core coated only with calix[n]arenes.

14. The metal-based nanomaterial according to claim 13, coated with an ultrathin layer of calix[n]arenes.

15. The metal-based nanomaterial according to claim 13, coated with an ultrathin layer of at least two types of calix[n]arenes.

16. The metal-based nanomaterial according to claim 13, wherein at least some grafted calix[n]arenes can be reacted with molecules or biomolecules.

17. The metal-based nanomaterial according to claim 13, wherein the metal is silver.

18. The metal-based nanomaterial according to claim 13, wherein the nanomaterial is coated with calix[4]arenes.

19. The metal-based nanomaterial according to claim 13, wherein the nanomaterial is bio-conjugated with a biomolecule, the biomolecule being bound to the calix[n]arene.

20. The metal-based nanomaterial according to claim 13 for use in immunoassays.

21. The metal-based nanomaterial according to claim 20, wherein the immunoassay is to detect anti-SARS-CoV-2 human IgG and/or anti-SARS-CoV-2 human IgM and/or the viral Protein S of SARS-CoV-2 human.
